(19) United States
(12) Patent Application Publication
(10) Pub. No.: US 2006/0015965 A1
(43) Pub. Date: Jan. 19, 2006

(54) Novel isoamylases and associated methods and products

(76) Inventors: Sadequr Rahman, Temperley St , Nicholls (AU); Christine Konik-Rose, Amaroo (AU); Matthew Morell, Aranda (AU); Ahmed Regina, Ngunnawal (AU)

Correspondence Address: Cooper & Dunham, 1185 Avenue of the Americas, New York, NY, 10036, US

(21) Appl. No.: 10/495,662

(22) PCT Filed: Nov. 12, 2002
(86) PCT No.: PCT/AU02/01539

§ 371 Date: Nov. 23, 2004

(30) Foreign Application Priority Data
Nov. 12, 2001 (AU) PR8827

(51) Int. Cl. C12Q 1/68; A01H 1/00; C12N 15/82

(52) U.S. Cl. 800/284; 435/6

(57) ABSTRACT

The present invention provides methods for distinguishing between different isoamylase genes of individual genomes of a polyploid plant. These methods enable a polyploid plant-to be produced and/or identified which has at least one impaired isoamylase gene. In particular, the methods of the invention enable various plants of a polyploidy species to be produced and/or indentified which, between the plants, lack a functional isoamylase gene for each genome of the plant. Such plants can be cross-bred to produce a plant which lacks any functional isoamylase protein. Furthermore, the invention provides numerous isoamylase gene, protein and cDNA sequences from various polyploidy plant species, including hexaploid wheat.

### FIELD OF THE INVENTION

The present invention relates to the identification of isoamylase mutants of polyploid plant species, and the identification and/or production of polyploid plants which do not produce a functional isoamylase. Furthermore, the present invention relates to isolated isoforms/variants of isoamylases from various polyploid plant species, in particular, genes of each isoamylase expressed in the endosperm of hexaploid wheat.

### BACKGROUND TO THE INVENTION

Starch is the predominant component of the cereal grain comprising about 65% of the dry weight at maturity. Starch is formed and accumulates predominantly in the endosperm tissue of the cereal grain. Starch also accumulates in potato tubers. Starch consists of two types of polymers of glucose: amylose and amylopectin which are present in a ratio of approximately 1:3. Amylose is essentially linear consisting of α-1,4 linked glucose residues but amylopectin is highly branched, containing branch points where the glucose residue is also linked in the 6 position to another series of α-1,4 linked glucose residues.

The biosynthesis of starch is a complex process. The first committed step in starch synthesis, the production of ADP-Glucose, may take place either inside or outside the amyloplast (Thorbjornsen et al., 1996) through the action of ADP-Glucose Phosphorylase (EC 2.7.7.27). The subsequent steps take place in the amyloplast but differ depending on whether amylose or amylopectin is synthesised. For the synthesis of amylose, the ADP-Glucose is acted on by granule bound starch synthase, GBSS (EC 2.4.18) which is also known as the waxy protein. For the synthesis of amylopectin the pathway is more complicated and based on studies of mutants as well as isolated enzymes, it has been proposed that ADP-Glucose is acted on by one or all of the three starch syntheses (EC 2.4.1.21) and the branching enzymes (EC 2.4.1.18). It has been hypothesised that this product is then acted on by debranching enzymes, which trim off inappropriate branches to produce the final amylopectin structure (Ball et al., 1996). There are two types of debranching enzyme activity, the isoamylase type and the pullulanase type and it has been suggested that both play a part in the synthesis of amylopectin (Kubo et al., 1999). In support of this model is the observation that the sugary-1 mutants of both rice and maize, which lack debranching enzyme activity, contain a highly branched polymer, phytoglycogen, which is normally not present (James et al., 1995, Kubo et al, 1999). Both pullulanase and isoamylase type activity is missing in rice mutants but the isoamylase gene in rice and the sugary-1 mutation map to chromosome 8 (Fujita et al., 1999) whereas the pullulanase gene maps to chromosome 4 (Nakamura, 1996). In maize the insertion of a transposable element in the gene described for isoamylase, SU1, leads to the sugary-1 phenotype, where phytoglycogen accumulates (James et al., 1995) and there is no amylopectin synthesis. However, in maize there is again a reduction in the amount of pullulanase in the endosperm.

Isoamylase genes have been isolated from monocotyledonous plants including maize (James et al. 1995), rice (Fujita et al. 1999), barley and wheat (Genschel et al. 2002, WO 99/58690, WO 99/14314). None of these publications compare multiple isoamylase gene sequences to identify polymorphisms in functional isoamylase genes in one polyploid species or use them to identify mutant plant cultivars with genes having impaired isoamylase function. Only single functional wheat isoamylase gene sequences were presented.

It is desirable to produce a plant which lacks a functional isoamylase gene, particularly because such plants should have high levels of free sugars (such as glucose). Considering the apprehension that surrounds the production and use of genetically modified plants, it is also desirable to produce non-transgenic cultivars of plants which lack the desired function, however, this process is particularly difficult for polyploid plants species (such as wheat). The present inventors have isolated and characterized gene sequences encoding the three isoforms of isoamylase from a hexaploid plant species. This has allowed the present inventors to devise screening, mutation and breeding strategies for ultimately obtaining a polyploid plant species lacking any functional isoamylase protein.

### SUMMARY OF THE INVENTION

The present inventors have identified polymorphisms in the genes which express the different isoforms of isoamylase from a polyploid plant species. These polymorphisms can be used to distinguish between the genomes of a polyploid plant species. In particular, these polymorphisms can be used to identify mutants of an isoamylase gene from a given genome of a polyploid plant which have a reduced production of a functional isoamylase protein when compared to a wildtype gene.

In a first aspect, the present invention provides a method of identifying a polyploid plant which has at least one impaired isoamylase gene, the method comprising screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase genes of said wildtype plants produce functional isoamylase in said storage organ.

The reduced production of a functional isoamylase protein from a particular isoamylase gene of the polyploid plant can be the result of a wide variety of reasons including, but not limited to, reduced or loss of gene transcription, partial or complete gene deletion, or the gene encodes a mutated protein with reduced or lacking isoamylase activity. In preferred embodiment, the impaired isoamylase gene does not express a functional isoamylase protein.

The polymorphism or mutation can be detected by any means known in the art. In a preferred embodiment, method comprises amplifying a region of an isoamylase gene. The amplification methodology may be any of a number of such systems well known in the art, eg polymerase chain reaction (PCR), strand displacement amplification assay (SDA), transcription-mediated amplification reaction (TMA), self-sustained sequence replication amplification reaction (3SR), and nucleic acid sequence replication based amplification reaction (NASBA), however, PCR is preferred.

In one embodiment, cDNA is reverse transcribed from mRNA obtained from the plant and analysed for the expression of a particular isoamylase gene. The lack of amplification product indicates that the gene is not expressed and/or not present in the plant analysed.

In another embodiment, an amplicon of different size is produced from each genome of the plant. These different amplicons correspond to individual isoamylase genes of the polyploid plant.

In a further embodiment, the method further comprises cleaving the amplicon(s), wherein the products of the cleavage are different sizes for each of the isoamylase genes of the plant. Preferably, the cleaving step is performed by a restriction enzyme. As the skilled addressee would be aware, such cleavage products can readily be analysed, for example, by agarose gel electrophoresis. Alternatively, the amplified fragment could be sequenced by standard techniques and the sequence data analysed to determine if the sequence contains any mutations that would result in the corresponding gene not being capable of the producing a functional isoamylase.

The lack of amplified product generally indicates that a given genome does not encode a functional isoamylase protein. However, there is a possibility that primers used in the amplification of the preferred embodiment of the method of the first aspect may not allow amplification due to the presence of a mutation where the primer hybridizes to the target nucleic acid. Accordingly, if no amplification product is obtained, it is preferred that the amplification reaction is repeated using a second set of primers that bind to the isoamylase gene at a region different to the first primer set. If the second set of primers produce an amplification product then this would imply that at least one of the primers of the first primer set did not bind due to a mutation. Accordingly, it would be preferable to further analyse the region to determine whether the genome of the polyploid plant species encodes a functional isoamylase protein. If the second primer set does not produce an amplification product then this would imply that the genome contains a large deletion and hence lack a gene encoding a functional isoamylase.

In a particularly preferred embodiment, the amplification is performed in the presence of an oligonucleotide primer comprising a sequence selected from: GGGGGTAAATCTTTTGGTCAGC (SEQ ID NO:1) or CTATTCTGGCTGTGGGAATACC (SEQ ID NO: 67) and/or CCTGGAACCTCTGGTCATTATG (SEQ ID NO:2).

In an alternate embodiment of the first aspect, the method further comprises contacting the plant, or protein containing material isolated from the plant, with an antibody that binds to at least one isoform, but not all isoforms, of an isoamylase from a polyploid plant, and determining the presence or absence of antibody bound protein. The lack of antibody binding typically indicates that at least one genome of the plant does not express a functional isoamylase.

Preferably, the antibody binds to the isoamylase protein at a region comprising a sequence selected from the group consisting of: TKAEDEDDDEEEAVA (SEQ ID NO:3), TKVEDEGEEDEPVA (SEQ ID NO:4), TLADLVTYNKKYN (SEQ ID NO:5), TLGDLVRYNNKYN (SEQ ID NO:6), FSPMPRSTSGG (SEQ ID NO:7), FSPMTRYTSGG (SEQ ID NO:8), GPILSFKGVD (SEQ ID NO:9), GPILSFRGVD (SEQ ID NO:10), VTEEVPLDPL (SEQ ID NO:11), VTEEVSLDPL (SEQ ID NO:12), FIEGELHNML (SEQ ID NO:13), FIEGELHDML (SEQ ID NO:14), PHCGHYLDVS (SEQ ID NO:15), PHCGHYLDIS (SEQ ID NO:16), WVTEMHVDGF (SEQ ID NO:17), and WVMEMHVDGF (SEQ ID NO:18).

Preferably, the antibody is detectably labeled. More preferably, the detectable label is selected from the group consisting of a radionucleotide, a fluorophore, a dye and a chemiluminescent molecule. Most preferably, an antibody is generated which is specific for each isoform of isoamylase and each of the antibodies are detectably labeled with a different label.

In a further preferred embodiment, the plant comprises a polymorphism or mutation in a region between introns 7 and 9, or a region between exons 1 and 3, of the isoamylase gene when compared to a wildtype sequence. More preferably, the region between introns 7 and 9 corresponds to between nucleotides 4681 and 5250 of SEQ ID NO:19. Considering this information, the skilled addressee could readily determine the corresponding regions from the other genomes of wheat, or the other genomes from different polyploid plant species.

Preferably, the polyploid plant is selected from the group consisting of: wheat, oats, potato, cotton, cherry, sugar-cane and bananas. In a further preferred embodiment, the plant is a cereal plant. Most preferably, the polyploid plant is wheat.

When the plant is a cereal it is preferred that the storage organ is a seed. When the plant is a potato plant it is preferred that the storage organ is a tuber. When the plant is cherry or banana, it is preferred that the storage organ is a fruit.

It is preferred that the method of the first aspect is used to detect naturally occurring impaired isoamylase genes of polyploid plants. However, in some circumstances it may be difficult to locate such a naturally occurring plant. In particular, in some circumstances it may be difficult locate the plants required to enable a polyploid plant to be bred which contains no functional isoamylases. Previously, the detection of a polyploid plant comprising at least one impaired isoamylase gene, but also comprising at least one functional isoamylase gene, could not be achieved as methods did not exist for suitably identifying each isoamylase gene from each genome of the polyploid plant. Considering the disclosure herein, this can now be achieved.

Thus, in a second aspect the present invention provides a method of producing and identifying a polyploid plant which has at least one impaired isoamylase genes, the method comprising;

i) exposing a polyploid plant, or a progenitor thereof, to conditions which promote DNA mutagenesis, and

ii) screening the plants obtained from step i), or progeny thereof, to identify plants which have at least one impaired isoamylase genes.

Preferably, the screening of step ii) is performed using the method of the first aspect.

In a third aspect, the present invention provides a method of producing and identifying a polyploid plant which has at least two impaired isoamylase genes, the method comprising;

i) identifying a polyploid plant which has at least one impaired isoamylase gene using the method according to the first aspect,

ii) exposing the plant, or a progenitor thereof, of step i) to conditions which promote DNA mutagenesis, and

iii) screening the plants obtained from step ii), or progeny thereof, to identify plants which have at least two impaired isoamylase genes.

Preferably, the screening of step iii) is performed using the method according to the first aspect.

In a fourth aspect, the present invention provides a method of producing and identifying a polyploid plant which has at least two impaired isoamylase genes, the method comprising;

i) exposing a first polyploid plant, or a progenitor thereof, to conditions which promote DNA mutagenesis,

ii) screening the plants obtained from step i), or progeny thereof, to identify plants which have at least one impaired isoamylase gene,

iii) crossing the plant of step ii) which has at least one impaired isoamylase gene with a second polyploid plant which has at least one impaired isoamylase gene, wherein the first and second plants have at least one impaired isoamylase gene which is on a different genome when compared to the other plant, and

iv) screening the plants obtained from step iii), or progeny thereof, to identify plants which have at least two impaired isoamylase genes.

Preferably, the screening of step ii) is performed using the method according to the first aspect.

Preferably, the screening of step iv) is performed using the method according to the first aspect.

Preferably, the progenitor is selected from, but not limited to, seeds, pollen or fruits.

In a fifth aspect, the present invention provides a method of producing a polyploid plant which has reduced levels of functional isoamylase, the method comprising;

i) crossing two parent polyploid plants each of which have at least one impaired isoamylase gene, and

ii) screening the progeny using the method of the first aspect to identify plants which produce reduced levels of functional isoamylase when compared to either of the parent plants,

wherein each of the parent plants have at least one impaired isoamylase gene which is on a different genome when compared to the other parent plant.

When a null mutant plant for each genome has been identified these individual plants can be crossed according to a method of the fifth aspect to ultimately provide a polyploid plant which does not produce a functional isoamylase protein.

Preferably, at least one of parent plants used in the cross is identified by the method of the first aspect.

Preferably, the screening of step ii) is performed using the method of the first aspect. However, at least in the instance where a plant is produced which lacks any functional isoamylase genes other methods of screening could be used. For example, an antibody which binds to each of the isoamylase isoforms could be used, wherein lack of antibody binding indicates the successful production of a polyploid plant lacking any functional isoamylase.

Preferably, at least one of the plants used in the cross comprises at least one impaired isoamylase gene that does not express a functional isoamylase protein.

With regard to the third to fifth aspects, the methods will typically comprise recovering F1 progeny plants from the cross, selfing the F1 plants, and selecting F2 plants which are homozygous for the impaired isoamylase genes at a given genome. With respect to the second aspect, the method will also typically comprise, after step ii), crossing the plant, or plant derived from the progenitor, with a second plant to produce F1 progeny, then selfing the F1 plants, and selecting F2 plants which are homozygous for the impaired isoamylase genes at a given genome.

The plant produced can be analysed to determine if starch synthesis has been altered when compared to the parent plants. Such analysis includes determining the levels of free sugars, as well as the proportions of amylose or amylopectin or phytoglycogen. Depending on the plant species, and the end product produced from the plant (e.g. cereals produced using wheat), the number of functional isoamylase genes (or lack thereof) required to produce the desired phenotype may vary.

Furthermore, the plant produced can be crossed with other cultivars with altered phenotypes. For example, these other cultivars may also have altered starch synthesis through the lack of at least one gene such as a gene encoding granule bound starch synthase or starch synthase II.

In some cases, particularly when the plant is a non-transgenic, the plant produced and/or identified by the methods of the invention may not have a suitable genetic makeup which is optimal for certain commercial activities or production under certain environmental conditions. In such cases it is desirable to use traditional plant breeding techniques to incorporate the impaired isoamylase gene onto a suitable genetic background.

Accordingly, in a sixth aspect the present invention provides a method of introducing an impaired isoamylase gene into the genome of a polyploid plant, the method comprising;

i) crossing a first parent plant with a second parent plant, wherein the second plant comprises at least one impaired isoamylase gene, and

ii) backcrossing the progeny of i) with plants of the same genotype as the first parent plant for a sufficient number of times to produce a plant with a majority of the genotype of the first parent but comprising the impaired isoamylase gene,

wherein progeny plants are screened for the impaired isoamylase gene.

Preferably, the screening is performed using the method according to the first aspect.

In polyploid wheat, the A and B genomes share a high degree of sequence similarity. The disclosure of the sequences for isoamylase genes from the A and B genome of polyploid wheat provides avenues of distinguishing between the isoamylase genes of the A and B genomes.

Accordingly, in a seventh aspect the present invention provides a method of distinguishing between the A and B genomes of wheat, the method comprising detecting a nucleotide sequence difference between the isoamylase gene of the A and B genomes, wherein the plant expresses isoamylase in the endosperm of developing grain.

The nucleotide sequence difference may be present anywhere in the isoamylase genes of the A and B genomes, including the intron sequences. However, in a preferred embodiment the nucleotide sequence difference occurs in a region between introns 7 and 9, or a region between exons 1 and 3, of the isoamylase gene when compared to a wildtype sequence

In an eighth aspect, the present invention a plant produced by the methods according to any one of the second to sixth aspects.

In a ninth aspect, the present invention provides a non-transgenic polyploid plant comprising at least two impaired isoamylase genes, wherein wildtype plants of the same species of polyploid plant accumulate starch in a storage organ of said wildtype plants, and wherein the corresponding isoamylase genes of said wildtype plants produce functional isoamylase in said storage organ.

Preferably, the plant is a cereal. More preferably, the cereal is wheat. The present inventors have used the method of the first aspect to identify two wheat varieties that do not produce at least one functional isoamylase isoform. Thus, in a particularly preferred embodiment, the wheat comprises impaired isoamylase genes in the B and D genomes. However, other embodiments of the invention include wheat which comprise impaired isoamylase genes in the A and B genomes, as well as wheat which comprise impaired isoamylase genes in the A and D genomes.

Most preferably, the plant is substantially lacking isoamylase activity in the endosperm during grain development.

In a tenth aspect, the present invention provides a storage organ of the plant according to the ninth aspect.

Preferably, the storage organ is a seed, tuber or fruit.

In an eleventh aspect, the present invention provides a food or non-food substance produced using a plant according to ninth aspect or the storage organ of the tenth aspect.

Preferably, the food substance is selected from the group consisting of bread, pasta, noodles, sponges, doughs and breakfast cereal.

As indicated above, the present inventors have found that the different isoforms of isoamylase produced by the isoamylase genes of a polyploid plant species may be distinguished by the production of antibodies that bind specifically to a particular isoform. Such antibodies are useful in screening varieties of a polyploid plant species for null mutants of a particular isoamylase isoform. Furthermore, the present invention enables the production of antibodies which would unequivocally bind to each isoamylase isoform. These antibodies are useful for the screening of plants which lack any functional isoamylase.

Thus, in a twelfth aspect, the present invention provides an antibody that binds to at least one isoform of an isoamylase from a polyploid plant.

In one embodiment, the antibody binds specifically to only one isoform of an isoamylase from a polyploid plant.

In another embodiment, the antibody binds specifically to all isoforms of isoamylase from a polyploid plant.

In a thirteenth aspect, the present invention provides a substantially purified polypeptide comprising an amino acid sequence selected from the group consisting of:

i) a sequence provided as SEQ ID NO:20,

ii) a sequence provided as SEQ ID NO:21,

iii) a sequence provided as SEQ ID NO:22,

iv) a sequence that is at least 93% identical to any one of i) to iii) which is at least 535 amino acids in length,

v) a sequence provided as SEQ ID NO:23,

vi) a sequence provided as SEQ ID NO:24,

vii) a sequence provided as SEQ ID NO:25, and

viii) a sequence which is at least 93% identical to any one of v) to vii).

In a preferred embodiment of the thirteenth aspect, the polypeptide is at least 95%, more preferably at least 97%, more preferably at least 98%, and most preferably at least 99% identical to a sequence provided in any one of SEQ ID NO's: 20 to 25.

Preferably, the polypeptide is substantially purified from wheat.

Preferably, the polypeptide has isoamylase activity.

The present invention also provides a polypeptide comprising a sequence encoded by any one of SEQ ID NO's 30 to 49, or a polypeptide at least 90%, more preferably at least 95%, more preferably at least 97%, more preferably at least 98%, and most preferably at least 99% identical thereto.

In an fourteenth aspect, the present invention provides a fusion protein comprising a polypeptide according to thirteenth aspect fused to at least one other polypeptide sequence

In a preferred embodiment of the fourteenth aspect, the at least one other polypeptide is selected from the group consisting of: a polypeptide that enhances the targeting and/or stability of the polypeptide of the thirteenth aspect and a polypeptide that assists in the purification of the fusion protein.

In a fifteenth aspect, the present invention provides an isolated polynucleotide comprising a nucleotide sequence selected from the group consisting of:

i) a sequence provided in SEQ ID NO: 50,

ii) a sequence provided in SEQ ID NO: 51,

iii) a sequence provided in SEQ ID NO: 52,

iv) a sequence encoding a polypeptide of the invention, and

v) a sequence which is at least 90% identical to any one of i) to iii) and which is at least 1710 nucleotides in length.

Preferably, the polynucleotide encodes a polypeptide which has isoamylase activity.

Preferably, the polynucleotide is at least 95%, more preferably at least 97%, and most preferably at least 99% identical to a sequence provided in SEQ ID NO's 50 to 52.

The present invention also provides a polynucleotide comprising a sequence provided as any one of SEQ ID NO's 30 to 49, or comprising an exon contained therein, or a polynucleotide at least 90%, more preferably at least 95%, more preferably at least 97%, more preferably at least 98%, and most preferably at least 99% identical thereto.

In a sixteenth aspect, the present invention provides a suitable vector for the replication and/or expression of a polynucleotide according to the fifteenth aspect. The vector may be, for example, a plasmid, virus or phage vector provided with an origin of replication, and preferably a promotor for the expression of the polynucleotide and optionally a regulator of the promotor. The vector may contain one or more selectable markers, for example an ampicillin resistance gene in the case of a bacterial plasmid or a neomycin resistance gene for a mammalian expression vector. The vector may be used in vitro, for example for the production of RNA or used to transfect or transform a host cell.

In a seventeenth aspect, the invention relates to host cells transformed or transfected with the vector of the sixteenth aspect.

Preferably, the host cell is a plant cell. More preferably, the plant cell forms at least part of a plant such as a seed.

In an eighteenth aspect, the present invention provides a plant transformed with an nucleic acid sequence according to the present invention, such that said nucleic acid sequence is capable of being expressed in said plant.

In a nineteenth aspect, the present invention the provides a method of altering starch synthesis in a plant, the method comprising disrupting the production and/or activity of a polypeptide according to the invention.

Preferably, the plant is wheat.

Preferably, starch synthesis is altered by increasing the amount of free sugars, or by altering the proportions of amylose or amylopectin or phytoglycogen, in the plant when compared to the same plant species which has not been exposed to the method of the nineteenth aspect.

As will be apparent, preferred features and characteristics of one aspect of the invention are applicable to many other aspects of the invention.

The terms “comprise”, “comprises” and “comprising” as used throughout the specification are intended to refer to the inclusion of a stated component or feature or group of components or features with or without the inclusion of a further component or feature or group of components or features.

The invention will now be described by way of the following non-limiting Figures and Examples.

### BRIEF DESCRIPTION OF THE DRAWINGS

FIG. 1. Southern blot analysis of the location of the sugary type isoamylase gene (wDBEI-D1) in wheat. DNA from the following Chinese Spring wheat-derived lines were loaded into the lanes as follows: Lane 1, CSN7AT7D (no chromosome 7A, four copies of chromosome 7D); lane 2, CSN7AT7B (no chromosome 7A, four copies of chromosome 7B); lane 3, CSN7DT7A (no chromosome 7D, four copies of chromosome 7A); lane 4, CSN7DT7B (no chromosome 7D, four copies of chromosome 7B); lane 5, CSN7BT7A (no copies of chromosome 7B, four copies of chromosome 7A); lane 6, CSN7BT7D (no copies of chromosome 7B, four copies of chromosome 7D) and lane 7 (euploid Chinese Spring). The size of the hybridising bands are indicated at the left. The probe was derived from a region of wheat isoamylase cDNA7 corresponding to exons 7 through to exon 14. Invariant bands probably represent the identical fragments from the three genomes.

FIG. 2. Deduced amino acid sequence of wheat isoamylase from the D genome (SEQ ID NO: 20). The nucleotide sequence of cDNA7 was translated to produce the deduced amino acid sequence. The putative signal (bold) and the putative N terminal (underlined) were predicted using the ChloroP program (Emanuelsson et al., 1999). The six isoamylase specific motifs (Beatty et al., 1999) are in italics and underlined, and are in the order V, VI, I, II, III and IV.

FIG. 3. Deduced amino acid sequence of a wheat isoamylase isoform. The nucleotide sequence of cDNA5 was translated to produce the deduced amino acid sequence (SEQ ID NO: 21). The putative signal (bold) and the putative N terminal (underlined) were predicted using the ChloroP program (Emanuelsson et al., 1999). The six isoamylase specific motifs (Beatty et al., 1999) are in italics and underlined and are in the order V, VI, I, II, III, IV.

FIG. 4. Deduced amino acid sequence of a wheat isoamylase isoform. The nucleotide sequence of cDNA9 (SEQ ID NO: 22) was translated to produce the deduced amino acid sequence. Part of the putative mature N terminus (underlined) was predicted using the ChloroP program (Emanuelsson et al., 1999). The six isoamylase specific motifs (Beatty et al., 1999) are in italics and underlined.

FIG. 5. Analysis of the expression of wDBE I-D1 in wheat endosperm. RNA from developing endosperm of indicated age was hybridised to a probe prepared from positions 4560 and 8530 of wDBEI-D1 (SEQ ID NO: 19) as described in the Materials and Methods.

FIG. 6. Comparison of the region between introns 7 and 9 of the wheat isoamylase genes. Polymorphisms are indicated. The consensus sequence is provided (SEQ ID NO:26), the A genome sequence (isoamyA) (SEQ ID NO: 27), the B genome sequence (isoamyB) (SEQ ID NO: 28), and the D genome sequence (isoamyD) (SEQ ID NO: 29).

FIG. 7. PCR amplification of wheat isoamylase gene using the primers Ariso8eF and Ariso9br followed by ScrF1/Bpm1 restriction digest distinguishes the three genomes in wheat. Lanes 1) T. durum, 2) T. tauschii, 3) Chinese Spring, 4) CSN7AT7B, 5) CSN7BT7A and 6) CSN7DT7B.

FIG. 8. Identification of two cultivars of wheat lacking the D and B genomes respectively. The null mutants were identified using the method as used for FIG. 7.

FIG. 9. Nucleotide sequence comparison for cDNAs 5 (isoamy5cDNA2) (SEQ ID NO:53), 7 (isoamy7cDNAD) (SEQ ID NO:54) and 9 (isoamy9cDNA3) (SEQ ID NO:55) for wheat isoamylase. The sequences were compared using the GCG PILEUP program (WebANGIS). Sequence polymorphisms are shown (boxed). The positions of the exons are shown as indicated, the exon segments are separated by double gaps. The 5′ untranslated region (5′-UTR) corresponds to nucleotide positions 1-101, the translation start ATG at 102-104, the coding region from nucleotides 102 to 2474, and the 3′-untranslated region (3′-UTR) from nucleotides 2475 to the end.

FIG. 10. Comparison of the three wheat isoamylase gene nudeotide sequences in the exon 8 to exon 9 region (SEQ ID NO's: 56 to 58). The sequences were aligned using PILEUP. Sequence polymorphisms are shown (boxed). The positions of the exon and intron boundaries are shown as indicated, the exon and intron segments are separated by double gaps. The nucleotide sequences correspond to nucleotides 4748-5129 of the A. tauschii genomic isoamylase sequence (SEQ ID NO:19).

FIG. 11. Isoamylase markers that distinguish the three isoamylase genes on the three genomes of wheat: PCR amplification with Exons 8-9 Forward (iso8F) and Reverse (Ariso9bR) primers followed by double restriction enzyme digest with Bpm1 and ScrF1. Lanes show fragments amplified from 1.wheat variety Chinese Spring; 2-4. Chinese Spring chromosome engineered wheat lines missing (null) for certain chromosomes: lane 2. null 7A lane 3. null 7B lane 4. null 7D 5-7. wheat lines lane 5. AUS17874 (wildtype), lane 6. null 7B AUS17519 (lacking 7B isoamylase gene marker), lane 7. AUS14113 (lacking 7D isoamylase gene marker) lane 8.100 bp marker. Labels to the left indicate genome specific fragments.

FIG. 12. Alignment (PILEUP) of three sequences (Q1B, Q1F, Q1H) obtained from a wild-type wheat line AUS17874 after amplification with exons 1-3 isoamylase primers (SEQ ID NO's: 59 to. 61). Comparison with sequences obtained from lines with null mutations for isoamylase revealed that Q1F was from the A genome, Q1B was from the D genome and Q1H was from the B genome of wheat. The sequences correspond to nucleotide sequence positions 2217-2707 of the A. tauschii genomic isoamylase sequence (SEQ ID No: 19).

FIG. 13. Left hand panel: PCR amplification of exons 1-3 region of the isoamylase genes performed with genomic DNA from durum wheat, oats and potato. Right hand panel: PCR amplification of exons 8-9 region of the isoamylase gene from genomic DNA from oats and durum.

FIG. 14. Alignment of five genomic nucleotide sequences obtained by PCR amplification of the region exons 1-3 of the oat isoamylase genes (SEQ ID NO's: 30 to 34).

FIG. 15. Alignment of two genomic nucleotide sequences obtained by PCR amplification of the region exons 1-3 of the durum wheat isoamylase genes (SEQ ID NO's: 35 and 36).

FIG. 16. Alignment of six genomic nucleotide sequences obtained by PCR amplification of the region exons 1-3 of the potato isoamylase genes (SEQ ID NO's: 37 to 42).

FIG. 17. Alignment of six genomic nucleotide sequences obtained by PCR amplification of the region exons 8-9 of the oat isoamylase genes (SEQ ID NO's: 43 to 48).

FIG. 18. Genomic nucleotide sequence obtained by PCR amplification of the region exons 8-9 of the durum wheat isoamylase genes (SEQ ID NO: 49).

### KEY TO SEQUENCE LISTING

SEQ ID NO: 1 and 2—PCR primers.
SEQ ID NO's: 3 to 18—Epitopes of wheat isoamylase isoforms.
SEQ ID NO: 19—Sequence of the isoamylase gene from Aegilops tauschii.
SEQ ID NO: 20—Sequence of isoamylase encoded by the D genome of wheat.
SEQ ID NO: 21—Sequence of an isoamylase isoform of wheat.
SEQ ID NO: 22—Partial sequence of an isoamylase isoform of wheat.
SEQ ID NO: 23—N-terminal sequence of SEQ ID NO: 20.
SEQ ID NO: 24—N-terminal sequence of SEQ ID NO: 21.
SEQ ID NO: 25—N-terminal sequence of SEQ ID NO: 22.
SEQ ID NO: 26—Consensus sequence of SEQ ID NO's 27, 28 and 29.
SEQ ID NO: 27—Sequence of a region between introns 7 and 9 of the A genome of wheat.
SEQ ID NO: 28—Sequence of a region between introns 7 and 9 of the B genome of wheat.
SEQ ID NO: 29—Sequence of a region between introns 7 and 9 of the D genome of wheat.
SEQ ID NO's: 30 to 34—5′ sequence of oat isoamylase genes.
SEQ ID NO's: 35 and 36—Partial sequence of durum wheat isoamylase genes.
SEQ ID NO's: 37 to 42—5′ sequence of potato isoamylase genes.
SEQ ID NO's: 43 to 48—Partial sequence of oat isoamylase genes.
SEQ ID NO: 49—Partial sequence of a durum wheat isoamylase gene.
SEQ ID NO: 50—Open reading frame of cDNA sequence encoding SEQ ID NO: 20.
SEQ ID NO: 51—Open reading frame of cDNA sequence encoding SEQ ID NO: 21.
SEQ ID NO: 52—Open reading frame of cDNA sequence encoding SEQ ID NO: 22.
SEQ ID NO: 53—cDNA sequence encoding SEQ ID NO: 21.
SEQ ID NO: 54—cDNA sequence encoding SEQ ID NO: 22.
SEQ ID NO: 55—cDNA sequence encoding SEQ ID NO: 20.
SEQ ID NO: 56—Sequence of a region between introns 7 and 9 of the A genome of wheat, larger region sequenced than SEQ ID NO:27.
SEQ ID NO: 57—Sequence of a region between introns 7 and 9 of the B genome of wheat, larger region sequenced than SEQ ID NO:28.
SEQ ID NO: 58—Sequence of a region between introns 7 and 9 of the D genome of wheat, larger region sequenced than SEQ ID NO:29.
SEQ ID NO: 59—Sequence of a region between exons 1 and 3 of the A genome of wheat.
SEQ ID NO: 60—Sequence of a region between exons 1 and 3 of the D genome of wheat.
SEQ ID NO: 61—Sequence of a region between exons 1 and 3 of the B genome of wheat.
SEQ ID NO's: 62 to 65—PCR primers.
SEQ ID NO: 66—Putative N-terminal sequence of a mature wheat isoamylase isoform.
SEQ ID NO: 67—PCR primer.

### DETAILED DESCRIPTION OF THE INVENTION

General Techniques

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (Editors), Current Protocols in Molecular Biology, Greene Pub. Associates and Wiley-Interscience (1988, including all updates until present) and are incorporated herein by reference.

Definitions

As used herein, the term “impaired isoamylase gene” indicates that the polymorphism or mutation of the gene ultimately results in “reduced production of a functional isoamylase protein”. The isoamylase gene can be impaired in any manner known in the art including, but not limited to, a frame shift altering the sequence of the encoded protein and/or resulting in a premature stop codon, reduced. RNA stability, partial or complete deletion of the gene, variation in the encoded protein sequence which results in a reduction or complete loss of isoamylase activity, a reduction or complete loss of promoter activity, insertion of a transposon or other element into the gene, and incorrect splicing to remove intron sequences during transcription. Accordingly, “reduced production of a functional isoamylase protein” incorporates situations where the level of protein produced by the gene is reduced (preferably abolished) and/or a protein is produced by the gene but the isoamylase activity is reduced (preferably abolished).

“Reduced” production of a functional isoamylase protein is generally used herein to refer to a lower level of functional isoamylase protein when compared to a wildtype plant. Preferably, at least 10% lower, more preferably at least 25% lower, more preferably at least 33% lower, more preferably at least 50% lower, more preferably at least 66% lower, more preferably at least 75% lower, more preferably at least 90% lower, more preferably at least 95% lower, and even more preferably at least 99% lower lower level of functional isoamylase protein when compared to a wildtype plant.

As used herein, the term “gene” means the deoxyribonucleotide sequences comprising the coding region of a structural gene and the including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences; these sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “polymorphism” as used herein denotes a variation in the nucleotide sequence between different isoamylase genes of individual plants. The polymorphisms may or may not result in a change in the expression of the functional isoamylase, for example, the nucleotide sequence change may be silent with regard to the phenotype. A “mutation” as used herein denotes a variation in the nucleotide sequence of an isoamylase gene in an individual plant compared to the sequence of the gene in wildtype plants, where the variation results in a discernable effect on the functionality of the encoded isoamylase, for example in its expression, activity or stability. Polymorphisms and mutations can be naturally occurring or induced, either directly or indirectly, by human intervention. As the skilled addressee would be aware, a mutation of a gene results in a polymorphism between to the mutated gene and the sequence from which it was derived. A polymorphic position is a predetermined nucleotide position within the sequence of the gene. In some cases, genetic polymorphisms are reflected by an amino acid sequence variation, and thus a polymorphic position can result in location of a polymorphism in the amino acid sequence at a predetermined position in the sequence of a polypeptide. Typical polymorphisms are deletions, insertions or substitutions. These can involve a single nucleotide (single nucleotide polymorphism or SNP) or two or more nucleotides.

A “deletion,” as used herein, refers to a change in either amino acid or nucleotide sequence in which one or more amino acid or nucleotide residues, respectively, are absent.

An “insertion” or “addition,” as used herein, refers to a change in an amino acid or nucleotide sequence resulting in the addition of one or more amino acid or nucleotide residues, respectively.

A “substitution,” as used herein, refers to the replacement of one or more amino acids or nucleotides by different amino acids or nucleotides, respectively.

A “null mutant” as used herein refers to the inability of a given genome of a polyploid plant species to produce a functional isoamylase. The term is also used in connection with a whole plant, wherein the plant is incapable of producing any functional isoamylase.

The term “corresponding” or “corresponds” when used in relation to a nucleotide or amino acid position indicates that the numbering of the polymorphism or mutation of interest may vary when compared to a specific position described herein, however, the skilled addressee could readily identify the relevant position in a related sequence. For instance, the sequences of isoamylase genes or proteins can be aligned, for example, by a computer program such as PILEUP (GCG package, WebANGIS) and “corresponding” positions/regions located.

By “linked” or “genetically linked” it is meant that a marker locus and a second locus are sufficiently close on a chromosome that they will be inherited together in more than 50% of meiosis, e.g., not randomly. Thus, the percent of recombination observed between the loci per generation (centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM apart and most preferably about 0 cM apart.

An isoamylase is generally referred to herein as a protein which removes side chains from a complex starch structure resulting in the production of amylopectin. Examples of such isoamylases are provided in SEQ ID NO:'s 20 to 22. The present invention also relates to orthologous isoamylases from other polyploid plant species, as well as mutants of any one of in SEQ ID NO:'s 20 to 22 which is at least 80%, more preferably at least 90%, more preferably at least 95%, and even more preferably at least 99% identical thereto. At least with regard to cereals, it is preferred that the isoamylase is at least expressed in the endosperm during early development of the grain.

Isoforms are different forms of the same protein or enzyme activity. For example, as used herein, isoforms of isoamylase correspond to the proteins encoded by the A, B and D genomes which have at least 93% sequence identity to Sequence IDs 20, 21 or 22.

Detection of Impaired Isoamylase Genes

Any molecular biological technique known in the art which is capable of detecting a polymorphism/mutation can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage, or a combination thereof (see, for example, Lemieux, 2000). The invention also includes the use of molecular marker techniques to detect polymorphisms closely linked to an impaired isoamylase gene. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al. (2001).

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells expressing, or that should be expressing, an isoamylase gene. However, it will generally be easier if PCR is performed on genomic DNA isolated from a plant.

A primer is an oligonucleotide, usually of about 20 nucleotides long, with a minimum of about 15 and a maximum of about 50 nucleotides, that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the induction of RE or catalytic nucleic add recognition/cleavage sites in specific target sequences. Primers may also contain additional sequences and/or modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al., eds., Short Protocols in Molecular Biology, 3rd ed., Wiley, 1995 and Sambrook et al., Molecular Cloning, 2nd ed., Chap. 13, Cold Spring Harbor Laboratory Press, 1989. Sequencing can be carried out by any suitable method, for example, dideoxy sequencing, chemical sequencing or variations thereof. Direct sequencing has the advantage of determining variation in any base pair of a particular sequence.

Hybridization based detection systems include, but are not limited to, the TaqMan assay and molecular beacons. The TaqMan assay (U.S. Pat. No. 5,962,233) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly. complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacon assay (U.S. Pat. No. 5,925,517). In the molecular beacon assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

Marker assisted selection is a well recognised method of selecting for heterozygous plants required when backcrossing with a recurrent parent in a classical breeding program. The population of plants in each backcross generation will be heterozygous for the gene of interest, normally present in a 1:1 ratio in a backcross population, and the molecular marker can be used to distinguish the two alleles. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed embryo rescue”, used in combination with DNA extraction at the three leaf stage and analysis for impaired isoamylase genes, allows rapid selection of desired plants, which may be nurtured to maturity in the greenhouse or field for subsequent further backcrossing to the recurrent parent.

Polypeptides

By “substantially purified polypeptide” we mean a polypeptide that has been at least partially separated from the lipids, nucleic acids, other polypeptides, and other contaminating molecules with which it is associated in its native state. Preferably, the substantially purified polypeptide is at least 60% free, preferably at least 75% free, and most preferably at least 90% free from other components with which they are naturally associated. Furthermore, the term “polypeptide” is used interchangeably herein with the term “protein”.

The % identity of a polypeptide is determined by GAP (Needleman and Wunsch, 1970) analysis (GCG program) with a gap creation penalty=5, and a gap extension penalty=0.3. Unless stated otherwise, the query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. Even more preferably, the query sequence is at least 100 amino acids in length and the GAP analysis aligns the two sequences over a region of at least 100 amino acids.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final protein product possesses the desired characteristics.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 30 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active or binding site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, a-amino isobutyric add, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino add analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

TABLE 1

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Polypeptides of the present invention can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Polynucleotides

By an isolated polynucleotide, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid molecule”.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides.

Polynucleotides of the present invention includes those which hybridize under stringent conditions to a sequence provided as SEQ ID NO's: 50 to 55, as well as sequences provided as SEQ ID. NO's: 30 to 49. As used herein, stringent conditions are those that (1) employ low ionic strength and high temperature for washing, for example, 0.015 M NaCl/0.0016 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Antibodies

The invention also provides monoclonal or polyclonal antibodies to polypeptides of the invention or fragments thereof. Thus, the present invention further provides a process for the production of monoclonal or polyclonal antibodies to polypeptides of the invention.

The term “binds specifically” refers to the ability of the antibody to bind to isoamylase but not other proteins obtained from the polyploid plant. In a preferred embodiment of the invention, the antibody can bind one isoamylase isoform, while not binding to the other isoforms within the polyploid plant, or any other protein.

As used herein, the term “epitope” refers to a region of an isoamylase isoform which is bound by the antibody. An epitope can be administered to an animal to generate antibodies against the epitope, however, antibodies of the present invention preferably specifically bind the epitope region in the context of the entire isoamylase protein.

If polyclonal antibodies are desired, a selected mammal (e.g., mouse, rabbit, goat, horse, etc.) is immunised with an immunogenic polypeptide. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

Preferably, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a colored or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin.

Vectors

One embodiment of the present invention includes a recombinant vector, which includes at least one isolated polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors of the present invention can direct gene expression in plant cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Host Cells

Suitable host cells to transform include any cell that can be transformed with a polynucleotide of the present invention. Host cells can be either untransformed cells or cells that are already transformed with at least one nucleic acid molecule (e.g., nucleic acid molecules encoding one or more proteins of the present invention). Host cells of the present invention either can be endogenously (i.e., naturally) capable of producing proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite, arthropod, animal and plant cells. Preferred host cells are plant cells.

Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Exemplary monocotyledons include wheat, barley, rye, triticale, oats, rice, and the like.

Wheat includes any species of Triticum, including Triticum aestivum (hexaploid), T. durum (durum wheat, also known as T. turgidum var. durum, tetraploid) and T. dicoccum (emmer wheat, tetraploid). Hexaploid wheat is understood to be Triticum aestivum. Einkom wheat (T. monococcum, diploid) is a species of wheat but plants of T. monococcum are not included in the invention as they are not polyploid.

Oats includes any species of Avena including Avena sativa. (cultivated oats, hexaploid) and Avena fatua (wild oats, hexaploid).

Potato as defined herein is any plant of the species Solanum tuberosum. Cotton as defined herein is any plant of the species Gossypium hirsutum or Gossypium barbadense.

Transgenic plants, as defined in the context of the present invention include plants (as well as parts and cells of said plants) and their progeny which have been genetically modified using recombinant DNA techniques. This would generally be to either i) cause or enhance production of at least one protein of the present invention in the desired plant or plant organ, or ii) disrupt the production and/or activity of a polypeptide of the present invention. Transformed plants contain genetic material that they did not contain prior to the transformation. The genetic material is preferably stably integrated into the genome of the plant. The introduced genetic material may comprise sequences that naturally occur in the same species but in a rearranged order or in a different arrangement of elements, for example an antisense sequence. Such plants are included herein in “transgenic plants”. A “non-transgenic plant” is one which has not been genetically modified with the introduction of genetic material by recombinant DNA techniques.

Several techniques exist for introducing foreign genetic material into a plant cell. Such techniques include acceleration of genetic material coated onto microparticles directly into cells (see, for example, U.S. Pat. No. 4,945,050 and U.S. Pat. No. 5,141,131). Plants may be transformed using Agrobacterium technology (see, for example, U.S. Pat. No. 5,177,010, U.S. Pat. No. 5,104,310, U.S. Pat. No. 5,004,863, U.S. Pat. No. 5,159,135). Electroporation technology has also been used to transform plants (see, for example, WO 87/06614, U.S. Pat. Nos. 5,472,869, 5,384,253, WO 92/09696 and WO 35 93/21335). In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during development and/or differentiation using appropriate techniques described herein.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Examples of plant promoters include, but are not limited to ribulose-1,6-bisphosphate carboxylase small subunit, beta-conglycinin promoter, phaseolin promoter, high molecular weight glutenin (HMW-GS) promoters, starch biosynthetic gene promoters, ADH promoter, heat-shock promoters and tissue specific promoters. Promoters may also contain certain enhancer sequence elements that may improve the transcription efficiency. Typical enhancers include but are not limited to Adh-intron 1 and Adh-intron 6.

Constitutive promoters direct continuous gene expression in all cells types and at all times (e.g., actin, ubiquitin, CaMV 35S). Tissue specific promoters are responsible for gene expression in specific cell or tissue types, such as the leaves or seeds (e.g., zein, oleosin, napin, ACP, globulin and the like) and these promoters may also be used. Promoters may also be active during a certain stage of the plants' development as well as active in plant tissues and organs. Examples of such promoters include but are not limited to pollen-specific, embryo specific, corn silk specific, cotton fiber specific, root specific, seed endosperm specific promoters and the like.

Under certain circumstances it may be desirable to use an inducible promoter. An inducible promoter is responsible for expression of genes in response to a specific signal, such as: physical stimulus (heat shock genes); light (RUBP carboxylase); hormone (Em); metabolites; and stress. Other desirable transcription and translation elements that function in plants may be used.

In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the cauliflower mosaic virus (35S and 19S) and the like may be used.

Generation of Mutant Plants

Polyploid plants which have at least one impaired isoamylase gene can be naturally occurring or,produced by technqiues which induce mutations in a plant. Techniques for generating mutant plant lines are known in the art. Examples of mutagens that can be used for generating mutant polyploid plants include irradiation and chemical mutagenesis. Mutants may also be produced by techniques such as T-DNA insertion and transposon-induced mutagenesis.

Chemical mutagens are classifiable by chemical properties, e.g., alkylating agents, cross-linking agents, etc. Useful chemical mutagens include, but are not limited to, N-ethyl-N-nitrosourea (ENU); N-methyl-N-nitrosourea (MNU); procarbazine hydrochloride; chlorambucil; cyclophosphamide; methyl methanesulfonate (MMS); ethyl methanesulfonate (EMS); diethyl sulfate; acrylamide monomer; triethylene melamine (TEM); melphalan; nitrogen mustard; vincristine; dimethylnitrosamine; N-methyl-N′-nitro-Nitrosoguani-dine (MNNG); 7,12 dimethylbenzanthracene (DMBA); ethylene oxide; hexamethylphosphoramide; and bisulfan.

An example of suitable irradiation to induce mutations is by gamma radiation, such as that supplied by a Cesium 137 source. The gamma radiation preferably is supplied to the plant cells in a dosage of approximately 60 to 200 Krad., and most preferably in a dosage of approximately 60 to 90 Krad.

Plants are typically exposed to a mutagen for a sufficient duration to accomplish the desired genetic modification but insufficient to completely destroy the viability of the cells and their ability to be regenerated into a plant.

Screening for Mutant Plants

A polymorphism that is specific for one of the isoamylase genes can be used as a marker to detect polyploid plants or lines that are impaired in the isoamylase gene. The polymorphism may lie within the gene or closely linked to it. The polymorphism may comprise only a single nucleotide change (single nucleotide polymorphism, SNP) which can be detected by standard sequencing methods or by the use of oligonucleotide primers which have the polymorphic nucleotides at or very close to the 3′ end of the primers. In this way, specific PCR amplification of one isoamylase gene segment can be performed.

Mutants that are impaired in at least one isoamylase gene function can be at the level of reduced expression of the gene. That is, assays such as reverse transcription-PCR (RT-PCR) assays can be used to identify plants that lack expression of one isoamylase gene, for example where the promoter of the gene is mutant but the coding region remains.

Polyploid plants substantially lacking the endosperm-expressed isoamylase activity or the storage organ-expressed isoamylase activity can be identified by a visual phenotype of the grain or storage organ, and thereforeeven rare mutants can be readily identified in a large population of wildtype grains or storage organs. For example, wheat grains substantially lacking isoamylase during grain development can be recognised by a shrunken, glassy or transluscent visual phenotype, as well as by their increased sugar and phytoglycogen levels and decreased amylopectin levels. This allows for a simple screening after mutagenesis of plants or seed already impaired in at least one isoamylase gene.

### EXAMPLES

### Example 1

### Materials and Methods

Plant Material

The Aegilops tauschii accession used, CPI 110799, has been previously shown in a survey to be most like the D-genome donor of wheat, based on the conservation of marker order (Lagudah et al., 1991).

Construction of Libraries

Construction of the genomic library from Aegilops tauschii has been described in (Rahman et al., 1997). Triticum aestivum cv Wyuna was used for the construction of the cDNA library and has been described before (Li et al., 1999).

Isolation of cDNAs

Based on alignment of protein and nucleotide sequences for isoamylase like debranching enzyme genes, primers were designed that were used to amplify a fragment, P1, from genomic wheat DNA. This fragment was sequenced and found to encode part of an isoamylase-like debranching enzyme sequence. The fragment was then used for the screening of a cDNA library from cv Rosella. The cDNA obtained (cDNA1) was incomplete and a probe based on the 5′ end of cDNA1 was used to screen a cDNA library prepared from wheat endosperm (cv Wyuna). The library was hybridised in 25% formamide, 2×SSC, 0.1% SDS at 42° C. with radiolabelled probe for 16 h and washed at 65° C. in 2×SSC, 0.1% SDS for 3 h. Three new cDNA clones were isolated.

Isolation of Aenomic Clones

The fragment P1 was used to screen a genomic library constructed from A. tauschii. From screening about 10⁶ clones, four positives (I1-I4) were isolated using the same hybridisation conditions are used for cDNA library screening. Clone I2 was used for further analysis.

DNA from chromosome engineered lines (Sears & Miller, 1985) was used for Southern analysis. Briefly, 10 μg of DNA was digested with BamHI and Dra I, electrophoresed on a 1% agarose gel and transferred to Hybond N+nylon membranes. The DNA was fixed using alkali and used for hybridisation with the indicated probes using the same conditions as described for cDNA library screening.

PCR amplification of the region between exons 1 and 3 was performed on the wheat lines AUS17874, AUS17519 and AUS14113 (Accession Nos. are for varieties in the Australian Winter Cereals Collection (AWCC), New South Wales Department of Agriculture, Tamworth, NSW, Australia). These wheat lines were found to be, respectively, containing the three isoamylase genes, null for the genome B isoamylase gene, and null for the genome D isoamylase gene (see Example 5). The PCR amplification used Hotstar Taq DNA polymerase, buffer as supplied with the enzyme, 2 mM dNTPs and 10 picomoles of each primer below. The primer sequences (5′ to 3′) were based on the A. tauschii isoamylase genomic sequence.

| | |
|---|---|
| E1-3 Forward primer: | |
| CTT CAC GCC AGA AGA TCT CAA G. | (SEQ ID NO:62) |
| E1-3 Reverse primer: | |
| CGT GCT ATA TGG AAG AGG GAT C. | (SEQ ID NO:63) |

PCR program:

95° C. for 5 mins

35 cycles of 95° C. for 30secs, 59° C. for 1 min, 72° C. for 2 mins

72° C. for 5 mins

25° C. for 1 min

10 μl of each of the 50 μl PCR reactions were electrophoresed on an agarose gel and a PCR product of approximately 500 bp was observed in each case. The remaining 40 μl PCR products were purified using the QIAquick PCR purification kit, and then ligated to pGEM-T vector using the Promega pGEM-T vector kit. The ligation mix was purified by isopropanol precipitation and the DNA resuspended in 15 μl water. 5 μl of each ligation mix was then used to transform E. coli strain DH5α competent cells by electroporation, and the cells plated onto LB agar containing ampicillin, IPTG and X-gal for blue (Lac+) and white (Lac-) colony screening. 15 white colonies were selected from each plate and grown in LB medium containing ampicillin, plasmid DNA prepared (miniprep), and the DNA inserts sequencing using BigDye reagents and methods.

Northern Hybridisation

Northern hybridisation was performed as described in (Li et al., 1999). Briefly, total RNA from indicated tissues was electrophoresed on a denaturing formamide/formaldehyde gel, blotted onto Hybond N+membrane and hybridised to indicated probes for 16 h at 42° C. in 25% formamide, 2×SSC and then washed at 65° C. in 2×SSC for 3×1 h and then exposed.

FISH

Aegilops tauschii and Triticum aestivum cv. Chinese Spring (common wheat) were used for FISH experiments. Multicolor FISH analysis was performed by using the phage clone I2. The pAs1 clone as used for the identification of A. tauschii chromosomes and the D genome chromosomes of common wheat (Rayburn & Gill, 1985). The isoamylase probes and pAs1 were labeled with digoxigenin-11-dUTP and biotin-16-dUTP, respectively, using a Nick Translation Kit (Roche Diagnostics). The details of hybridization and detection in the FISH experiment have been described by Fukui et al. (2001), Rahman et al. (1997) and Turner et al. (1999).

Screening Exons 7 to 9 Isoamylase Null Mutants

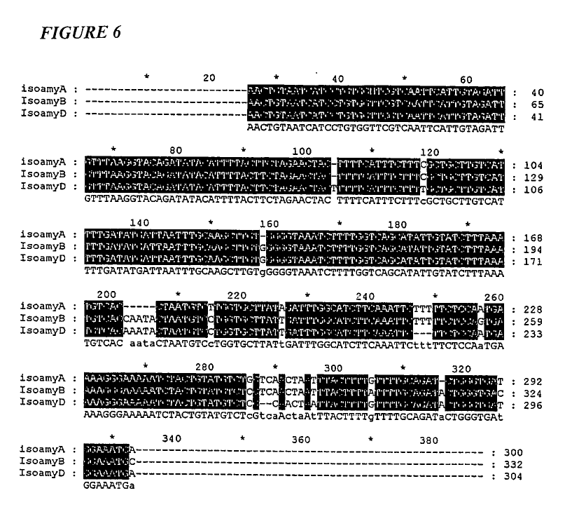

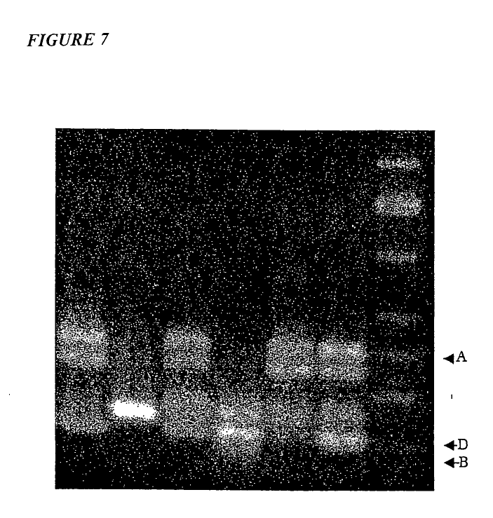

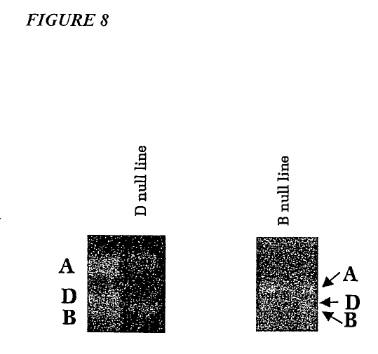

Based on the sequence from the isoamylase gene from A. tauschii several primer pairs were designed targeting the 5′ region of the gene. Three polymorphic sequences were obtained from the intron 8 region of the wheat gene (FIG. 6). Restriction site variation among the three sequences were exploited to design markers that could distinguish the three genomes in wheat. After PCR amplification the resulting PCR products were cleaved by a double digestion with the restriction enzymes ScrF1 and Bpm1 yielding a 220 bp fragment from the A genome, 150 bp fragment from the B genome and a 170 bp fragment from the D genome (FIG. 7).

### Example 2

### Isolation of an Isoamylase Gene from A. tauschii

Based on an alignment of isoamylase cDNA sequences from maize and rice, primers L (GAGGTGATCATGGATGTTGTCTT) (SEQ ID NO: 64) and R (AGTATAGATGCAAGGTCAAAGCA) (SEQ ID NO: 65) were designed to amplify the corresponding gene from the wheat genome. A product of approximately 250 bp was amplified from the wheat genome. The identity of the product, called P1, was determined by sequencing and it was clear that an isoamylase type sequence had been amplified.

The insert P1 was used to screen a lambda genomic library constructed from A. tauschii, the donor of the D genome to wheat. Four clones were isolated and purified. On restriction digestion followed by hybridisation it was clear that the same gene was contained in each of the four clones, I1 to I4. The inventors have called this gene wDBE I-D1.

The gene sequence for wDBE I-D1 is shown in SEQ ID No: 19. The sequence comprises 9643 nucleotides, which corresponds to the 9306 nucleotides of SEQ ID No: 1 of Australian Provisional Patent application No. PR8827, with an additional 48 nucleotides in intron 4 and an additional 289 nucleotides at the 3′ end. The coding region of wDBE I-D1 extends from the ATG at position 1867-1869 to the TGA stop codon at position 9631-9633 with reference to SEQ ID No: 19. The gene consists of 18 exons which vary in size from 71 bp to 500 bp. The overall length of the gene covering the coding sequences is approximately 7.7 kb. All the introns possessed the conserved 5′ GT and 3′ AG nucleotides. The start and end positions of the exons are provided in Table 2.

TABLE 2

| Exon | Start | End | Exon | Intron |
|---|---|---|---|---|
| number | position | position | size | size |
| 1 | 1766 | 2241 | 476 | 101 |
| 2 | 2342 | 2524 | 183 | 91 |
| 3 | 2615 | 2707 | 93 | 309 |
| 4 | 3016 | 3168 | 153 | 240 |
| 5 | 3408 | 3484 | 77 | 877 |
| 6 | 4361 | 4502 | 142 | 72 |
| 7 | 4574 | 4681 | 108 | 101 |
| 8 | 4782 | 4867 | 86 | 239 |
| 9 | 5106 | 5177 | 72 | 73 |
| 10 | 5250 | 5376 | 127 | 230 |
| 11 | 5606 | 5692 | 87 | 945 |
| 12 | 6637 | 6729 | 93 | 837 |
| 13 | 7566 | 7724 | 159 | 749 |
| 14 | 8473 | 8556 | 84 | 212 |
| 15 | 8768 | 8848 | 81 | 83 |
| 16 | 8931 | 9010 | 80 | 133 |
| 17 | 9143 | 9260 | 118 | 116 |
| 18 | 9376 | 9639 | 268 | |

### Example 3

### Location of the Wheat Homologue of wDBEI-D1.

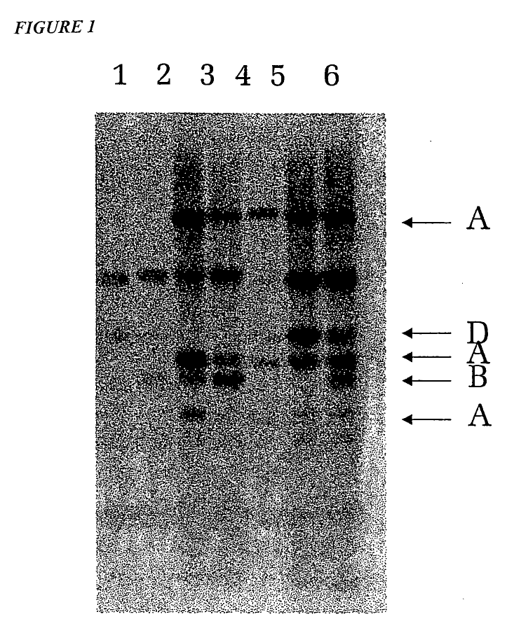

DNA was isolated from chromosome engineered lines of wheat cultivar Chinese Spring missing one of chromosomes 7A, 7B and 7D. The DNA was digested with the enzyme Bam HI and Dra I, electrophoresed and blotted. A probe from exons contained within positions 4560-8510 of wDBE I-D1 (part of exon 7 through to exon 14) was used to probe the blot. It was clear that fragments of approximate sizes 3.5 kb, 1.5 kb and 0.7 kb could be assigned to the A genome (FIG. 1). A fragment of 1.4 kb could be assigned to the B genome and a fragment of 1.7 kb could be assigned to the D genome. In addition there were fragments of 2.5 kb and 0.5 kb that could not be assigned to any chromosome. Fragments smaller than 500 bp were not retained in the gel used for the blot. The assignments of fragments of size 3.5 and 1.5 kb to the A genome, 1.7 kb to the D genome and 1.4 kb to the B genome was confirmed using an identical combination of enzymes and different probe comprising of exon sequences contained between positions 4525-5129 of wDBE I-D1 (data not shown). The assignment of a 1.7 kb fragment to the D genome is consistent with the sequence information obtained for wDBE I-D1.

FISH

To confirm the physical location of wDBE I-D1 to chromosome 7, FISH was carried out with the clones I1-I4. No discrete localised signals were obtained, probably due to the presence of repetitive sequences in the genomic clones. However, by using clone I2 and high Cot DNA as competitor, clear signals could be observed at the short arm of chromosome 7D (data not shown). The location of the gene on chromosome 7 was also established by PCR using deletion stocks of cv Chinese Spring (data not shown).

### Example 4

### Isolation of cDNAs for Isoamylase from Wheat Endosperm Library

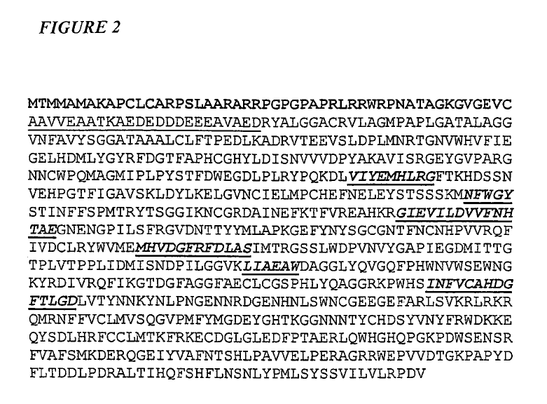

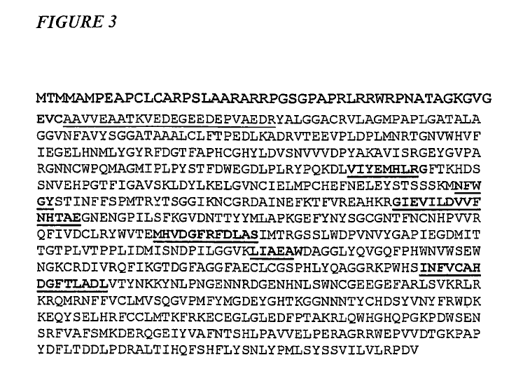

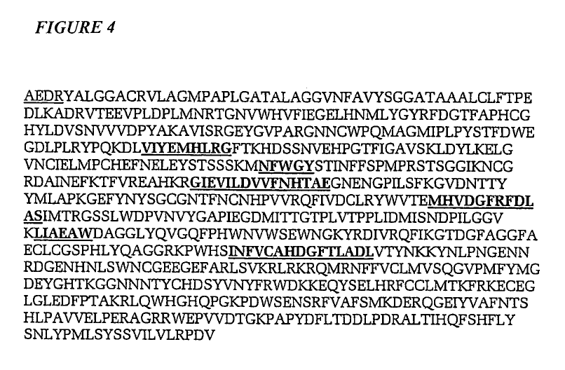

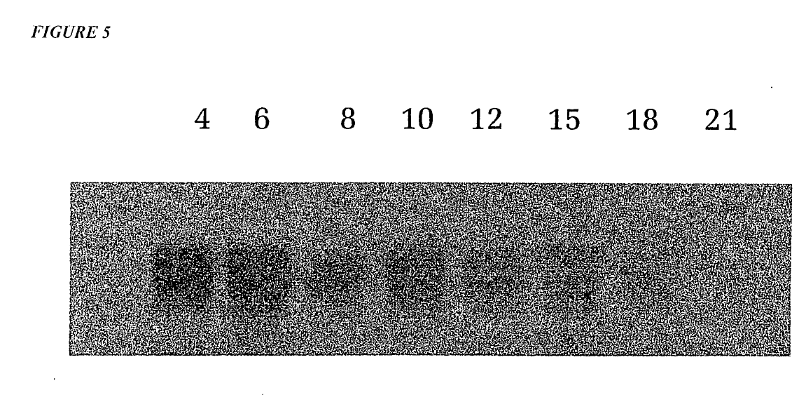

PCR fragment P1 was labelled radioactively and used as a probe to screen a cDNA library constructed from the wheat cultivar Rosella. A partial cDNA (termed cDNA 1) of 1800 bp was isolated. The sequence of cDNA1 from position 90 to 280 was amplified using primers specific for the region and radiolabelled. The amplified fragment was used as a probe to isolate three larger cDNAs (cDNAs 5,7 and 9) from a cDNA library constructed from the cultivar Wyuna. The deduced amino acid sequences encoded by cDNA7, cDNA5, and cDNA9 are presented in FIGS. 2, 3 and 4 respectively. cDNAs 7 and 5 differ by 20 amino acids. The cDNAs 7 and 9 differ in 12 positions and cDNAs 5 and 9 by 3 substitutions but cDNA 9 is not full length and so there may be further differences. The results are consistent with cDNAs 5, 7 and 9 representing products of the three wheat genomes.

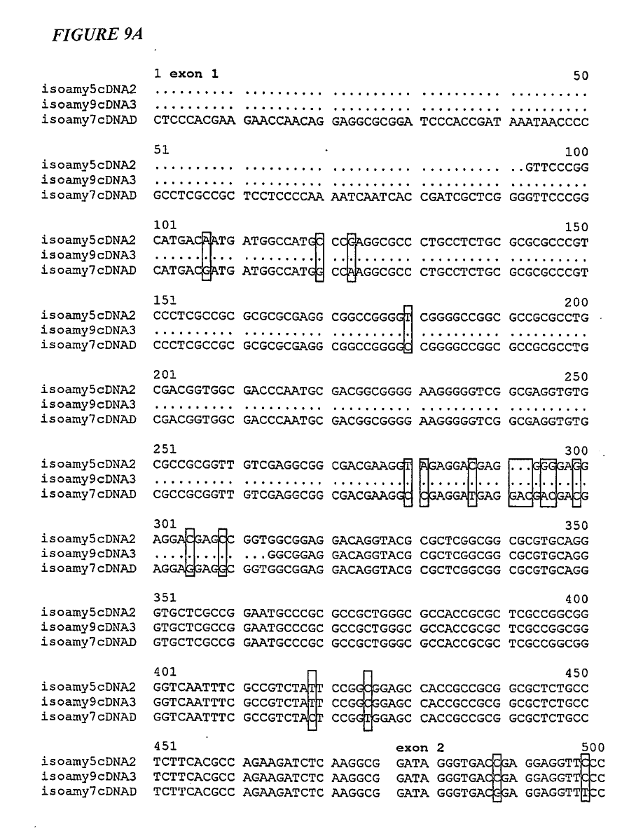

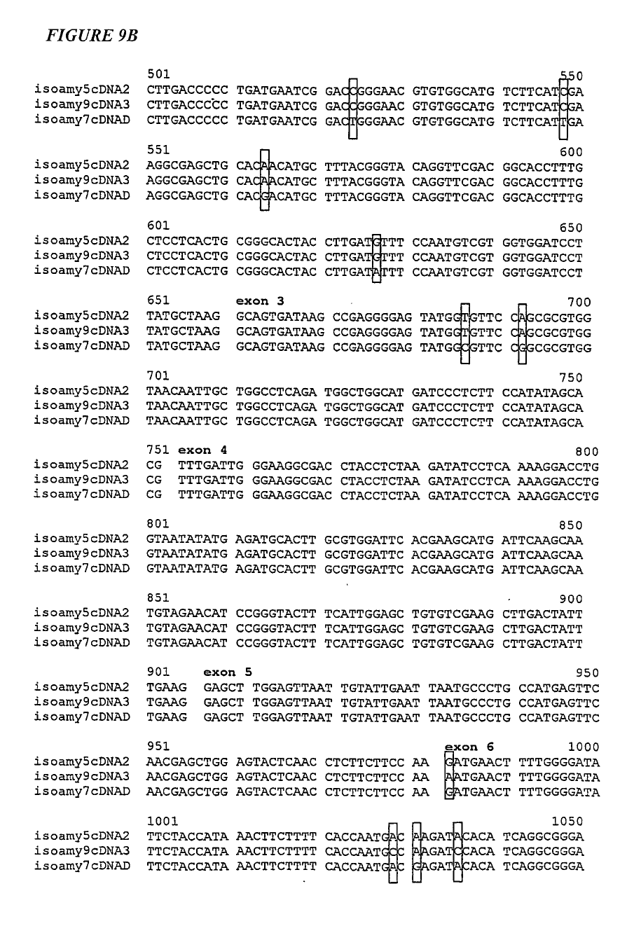

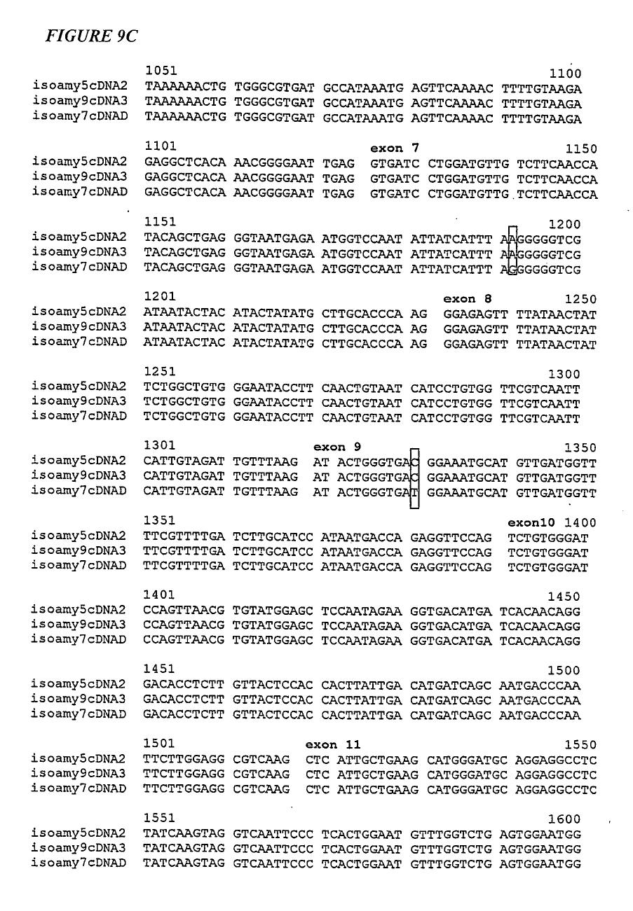

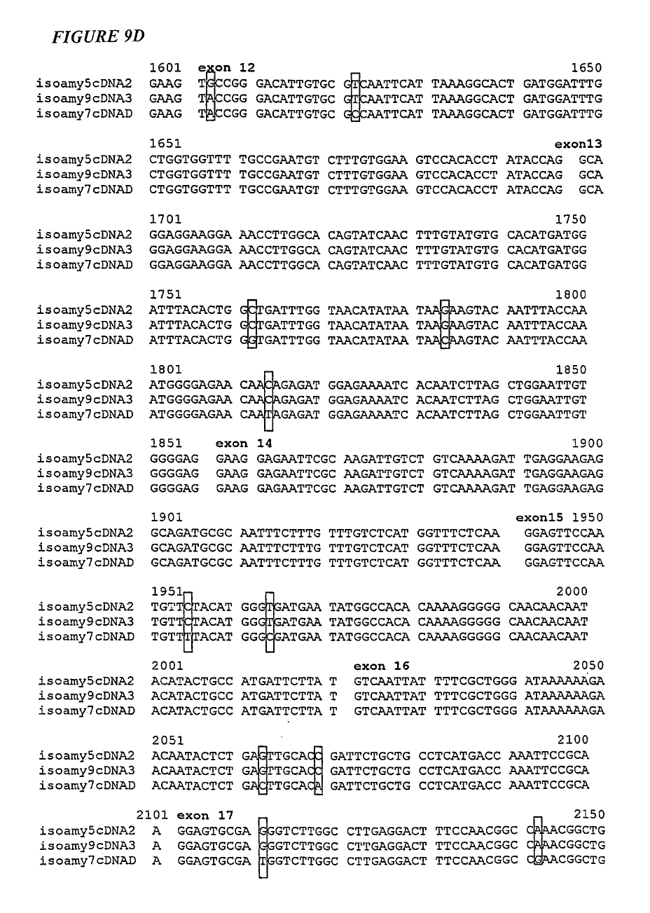

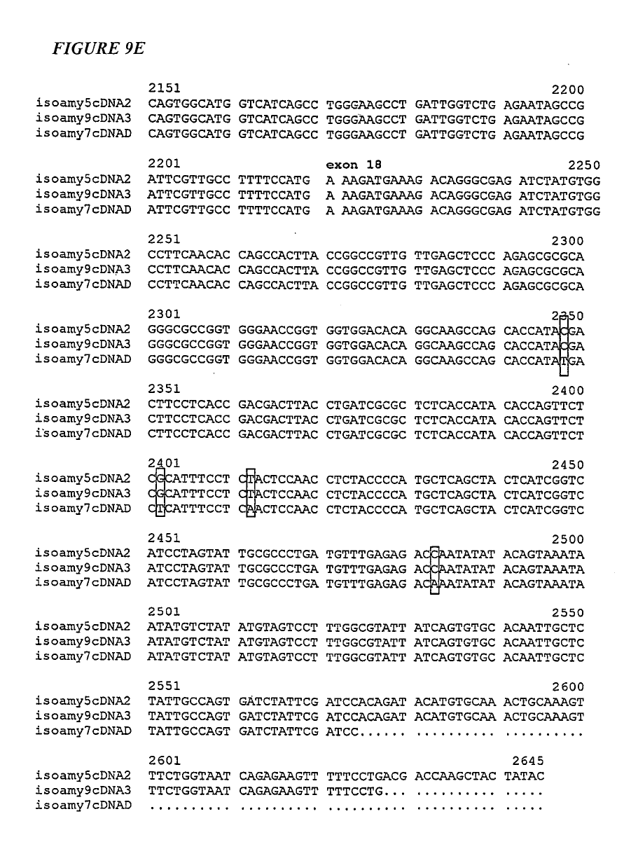

The nucleotide sequences for cDNAs 5, 7 and 9 are shown and compared in FIG. 9. Sequence polymorphisms were present throughout the gene sequences, but were more concentrated in the 5′-UTR and 3′-UTR. The sequence for cDNA 7, corresponding to the isoamylase gene from the D genome of wheat, was more polymorphic than the other two, which were identical except for polymorphisms at positions 983, 1029 and 1036, all within the exon 6 region of the cDNAs, and position 1606 within exon 12 with reference to FIG. 9. Two of these polymorphisms result in restriction enzyme site polymorphisms, at position 1033-1036 (GATC vs GATA, restriction enzyme Sau3A) and position 1604-1607 (GTAC vs GTGC, Rsal) which could readily be converted to a molecular marker, for example using reverse transcription-PCR (RT-PCR) from RNA from wheat lines, using flanking primers, followed by restriction digestion with Sau3A.

Full length sequences for the cDNA corresponding to cDNA 9 or others can readily be obtained by techniques well known in the art such as 5′ RACE (rapid amplification of cDNA ends).

Beatty et a., (1999) identified two motifs (motif V and VI) that occurred in all debranching enzymes in addition to the four regions (motifs I-IV) that were found to be present in all members of the α-amylase superfamily of starch hydrolytic enzymes (Jesperson et al., 1993). Five of these six motifs occur in the deduced sequence from cDNA 7 and are indicated (FIG. 2). The putative N-terminal is underlined and the putative transit sequence (Emanuelsson et al., 1999) is in bold. The mass of the putative mature peptide is 83 kDa.

Analysis of the wheat sequence using the ChloroP program (Emanuelsson et al., 1999) leads to a 50 amino acid transit peptide and a predicted N-terminal sequence of AAVVEAATKAEDEDDDDEEE (SEQ ID NO: 66). The predicted molecular size of the mature protein is again 83 kDa.

Northern Hybridisation of Wheat Isoamylase RNA

A probe consisting of exons contained within nucleotide positions 4560 to 8530 of wDBE I-D1 (part of exon 7 through to 14) was used to investigate the expression of hybridising RNA. The results are shown in FIG. 5. An RNA molecule of approximately 2900 bp was found to hybridise. The transcript could not be detected 4 days after anthesis but from 6 days after anthesis the amount of transcript appeared to be at the maximal level. This pattern of expression is in sharp contrast to the accumulation of SBE I (Rahman et al., 1999) and is more like the accumulation pattern of transcripts for SBE Ha (Rahman et al., 1999). The amount of hybridising RNA detected declined from 18 days after anthesis.

Northern hybridisations reveals that the isoamylase gene is expressed from early on in grain development in wheat and declines towards the end of grain filling. This result is similar to that of the expression pattern of SBE IIa, SSI and SSII. The expression of SSIII is somewhat earlier and that of SBE I is later. These results suggest that the structure of the amylopectin could change during grain filling.

### Example 5

### Screening Isoamylase Null Mutants

Based on the sequence from the isoamylase gene from A. tauschii, several primer pairs were designed to amplify regions from the three T. aestivum (A, B and D genome) isoamylase genes. Amplification of the intron 8 region of the genes by PCR using the primer pair (GGGGGTAAATCTTTTGGTCAGC) (Ariso8eF) (SEQ ID NO: 1) and (CCTGGAACCTCTGGTCATTATG) (Ariso9bR) (SEQ ID NO: 2) generated fragments which were cloned and sequenced. Three distinct sequences were obtained and were presumed to be derived from the A, B and D genomes. The sequences were compared by PILEUP (FIG. 6). Restriction site polymorphisms were evident which allowed the generation of restriction enzyme-based markers. PCR amplification of the wheat segments with the primer pair Ariso8eF (SEQ ID NO: 1) and Ariso9bR (SEQ ID NO: 2) generated fragments which were digested with the restriction enzymes Scrf1 and Bpm1, yielding a 220 bp fragment from the A genome, a 150 bp fragment from the B genome and a 170 bp fragment from the D genome which could be readily separated by gel electrophoresis (FIG. 7). The correspondence of these fragments with the particular genomes was accomplished by screening the euploid and chromosome engineered Chinese Spring lines lacking the 7A, 7B or 7D chromosomes for the same markers.

This approach using these molecular markers was used to screen wheat varieties to identify null mutations in the three genomes. Two wheat varieties (Accession Nos AUS17519 and AUS14113. Accession Nos. are for varieties in the Australian Winter Cereals Collection (AWCC), New South Wales Department of Agriculture, Tamworth, NSW, Australia) were each lacking one of the genome specific markers. AUS17519 was lacking the B genome-specific marker, while AUS14113 lacked the D genome-specific marker (FIG. 8). These lines are therefore null mutants for the B genome and D genome isoamylase genes, respectively.

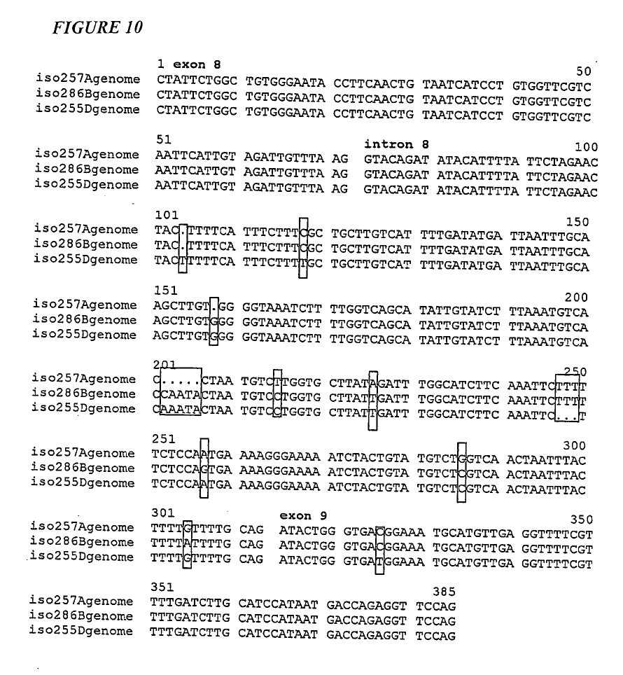

An extended region was amplified using a different forward primer: iso8F: CTA TTC TGG CTG TGG GAA TAC C (SEQ ID NO: 67). This primer corresponds to a sequence in the exon region rather than the intron region (for Ariso8eF) and can therefore be used on both cDNA and genomic DNA. This set of primers (iso8F and Ariso9bR) along with the double restriction enzyme digest worked just as well as the previous markers at distinguishing the three isoamylase genes on the A, B and D genomes of wheat. The three sequences were determined by cloning and sequencing the amplified fragments and are compared in FIG. 10. The sequenced regions include part of axon 8, intron 8 and all of exon 9. The genome identity of each sequence was determined by comparison of the sequences with those in FIG. 6 as well as with the D genome cDNA 7. FIG. 10 shows an alignment of the nucleotide sequences for this region corresponding to the A (iso257Agenome), B (iso286Bgenome) and D (iso255Dgenome) genome isotypes.

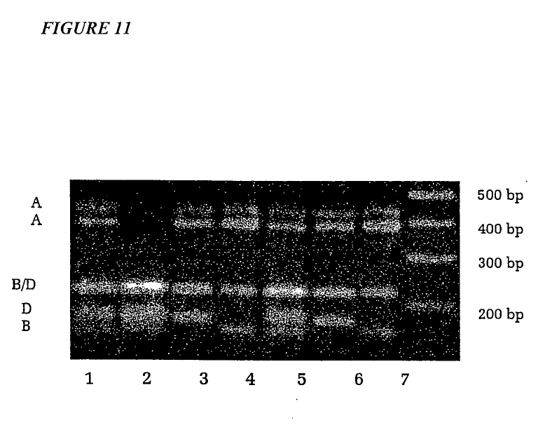

Wheat lines including the euploid and chromosome engineered Chinese Spring lines were tested for the markers, using PCR amplification with primers iso8F and Ariso9bR followed by digestion with the restriction enzymes Bpm1 and ScrF1. Gel electrophoresis (FIG. 11) showed the absence of the B genome-specific marker in AUS17519 and the D genome-specific marker in AUS14113, confirming the results described above and indicate that these are null lines for one of the isoamylase genes.

Approximately 2000 wheat lines from the AWCC germplasm collection were screened with the isoamylase markers, looking for further lines that are missing one or more of the isoamylase markers and therefore null for one of the isoamylase genes. Three additional wheat lines to those described above were null for the B genome-specific isoamylase gene (AUS19243, AUS19253, and AUS19249) and one additional wheat line was null for the D genome-specific isoamylase gene (AUS17683). Further screening is performed to identify an A genome-specific null line.

These lines are crossed to produce wheat lines lacking both the B and D genome specific isoamylase genes. A line lacking the A genome-specific gene is then crossed with the BD double null line to produce a triple null line for isoamylase, that is, substantially lacking isoamylase expression in the endosperm of the grain.

### Example 6

### Identification of Markers in the Exon 1-Exon 3 Region

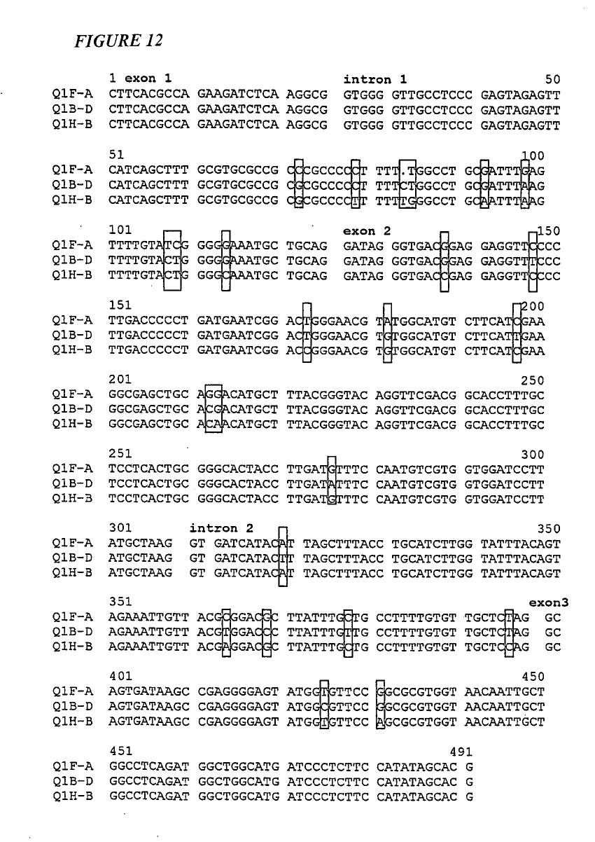

The exon 1 to exon 3 region of the isoamylase genes was amplified from wildtype wheat line AUS17874 using the primers iso1F and iso1R as described in Example 1. Cloning and sequencing of the amplified fragment showed three distinct sequences which are aligned in FIG. 12. The same amplification, cloning and sequencing procedure was performed for lines AUS17519 and AUS14113, which were shown as described above to be null for the B genome and D genome isoamylase genes, respectively.

Analysis of the nucleotide sequences from exons 1 to 3 (FIG. 12) using the GCG program (WebANGIS) showed three different sequences (Q1F, Q1B and Q1H) present in the AUS17874 (wild type, WT) wheat line and two different isotypes for each of the AUS17519 (null B) and AUS14113 (null D). From these results it was possible to assign the three isoamylase gene isotypes to the three wheat genomes, as follows. Nucleotide sequence Q1B was present in AUS17874 and AUS17519 but lacking in AUS14113 and so corresponds to the D genome isotype. The sequence of Q1B is also consistent with the corresponding regions of the exons in cDNA 7, confirming its genome identity. Nucleotide sequence Q1H was present in AUS17874 and AUS14113 but lacking from AUS17519 and so corresponds to the B genome isotype. Nucleotide sequence Q1F was present in all three wheat lines and therefore corresponds to the A genome isotype.

Polymorphisms occur throughout the sequenced regions (FIG. 12). Markers to detect one or more of these polymorphisms can be developed for this region of the isoamylase gene (exons 1 to 3) in order to distinguish the three isotypes found on the three wheat genomes, as described above for the region between exons 8 and 9. For example, restriction site polymorphisms are present at positions 70-75 (GCGCGC vs GCCCGC, restriction enzyme BssHII), 96-99 (TTAA vs TTGA, restriction enzyme Msel), and 105-108 (GTAC vs GTAT, restriction enzyme Rsal) with reference to the sequences shown in FIG. 12. Primers flanking any of these polymorphisms can be used to amplify a region spanning them, and the amplified products analysed by restriction digestion and gel electrophoresis to detect null mutants.

### Example 7

### Durum Wheat, Oats and Potato Isoamylase Genes

Durum wheat (Triticum turgidum), oats (Avena sativa) and potato (Solanum tuberosum) are also polyploid plants and accumulate starch in grain or tubers. The methods of the invention as described above for wheat can also be used to distinguish isoamylase gene isotypes in these plants and therefore be used to identify null mutants in one or more isoamylase gene isotypes. This will lead to the production of non-transgenic durum wheat, oats and potato lacking multiple isoamylase isotypes, either by combining the null mutations in one plant line or the use of mutagenesis to inactivate one remaining isoamylase gene. This will provide modified starch and starch products from these plant sources.

Two regions of the isoamylase genes of durum wheat, oats and potato (exons 8-9 and exons 1-3) were amplified by PCR. Total (genomic) DNA from was obtained from durum wheat, oats and potato plants. The PCR amplification used Hotstar Taq DNA polymerase, the buffer supplied with the enzyme, 2 mM dNTPs and 10 picomoles of each primer below (5′ to 3′). The primer sequences were based on the A. tauschii genomic sequence. For the exon 1-3 region,

| | |
|---|---|
| E1-3 Forward primer (iso1F): | |
| CTT CAC GCC AGA AGA TCT CAA G | (SEQ ID NO:62) |
| E1-3 Reverse primer (iso1R): | (SEQ ID NO:63) |
| CGT GCT ATA TGG AAG AGG GAT C | |
| For the axon 8-9 region, | |
| E8-9 Forward primer (iso8F): | |
| CTA TTC TGG CTG TGG GAA TAC C | (SEQ ID NO:67) |
| E8-9 Reverse primer (iso9bR): | |
| CCT GGA ACC TCT GGT CAT TAT G | (SEQ ID NO:2) |

PCR program:

95° C. for 5 mins

35 cycles of 95° C. for 30secs, 59° C. for 1 min, 72° C. for 2 mins

72° C. for 5 mins

25° C. for 1 min

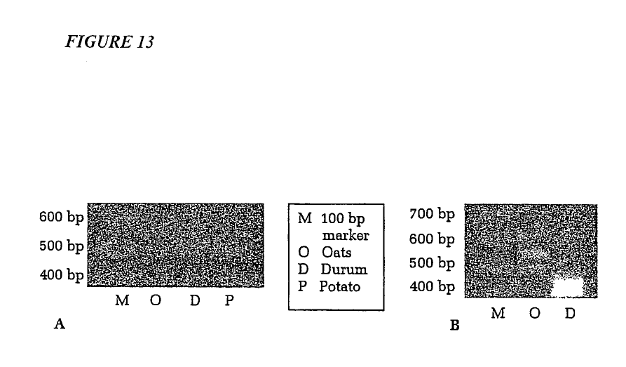

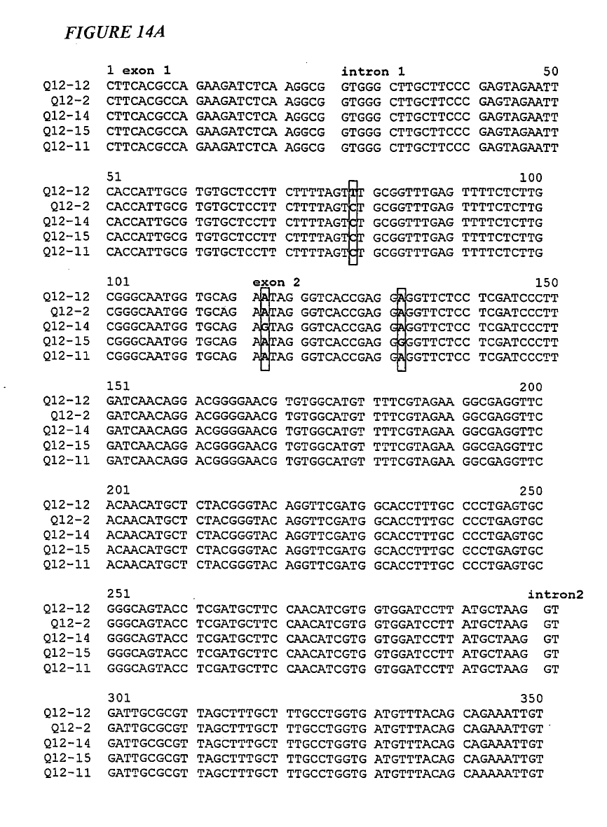

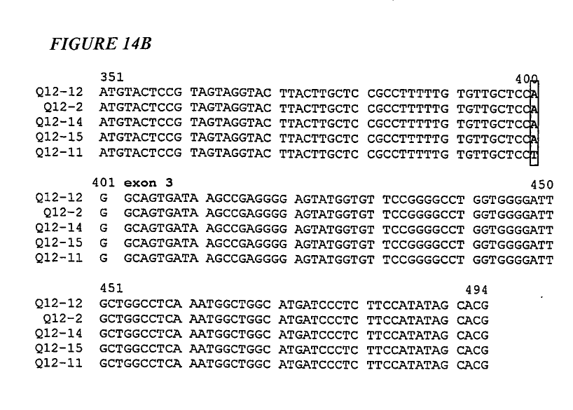

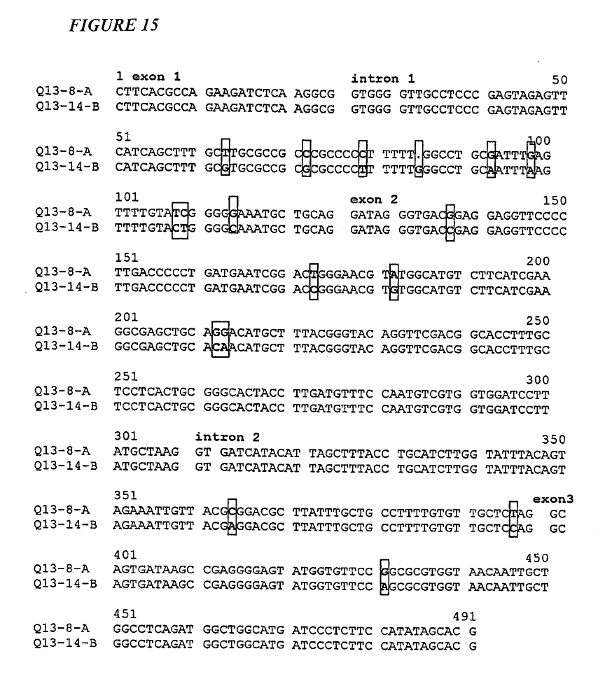

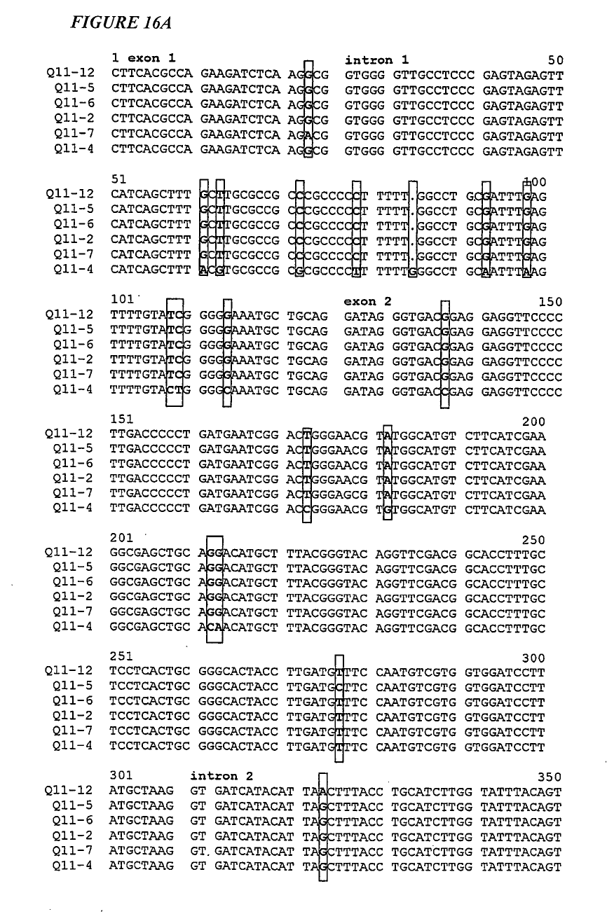

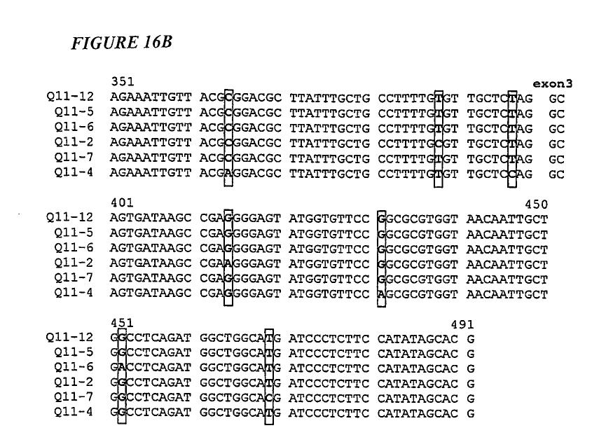

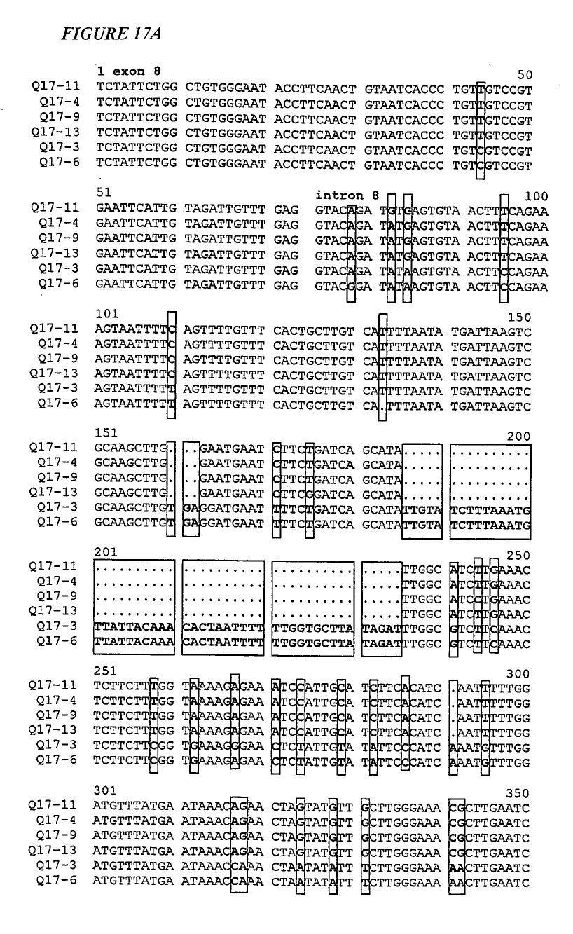

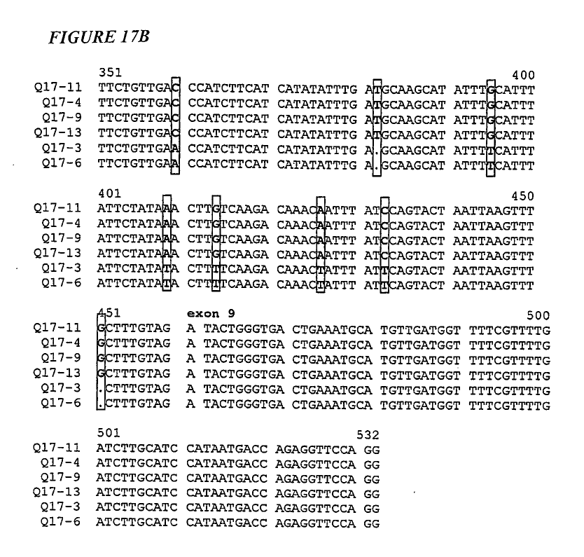

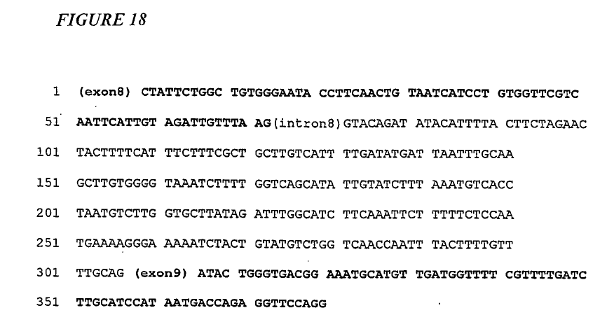

10 μl of each of the 50 μl PCR reactions were run out on an agarose gel to check for a PCR product. The remaining 40 μl of PCR products were purified using the QIAquick PCR purification kit, then ligated to pGEM-T vector using the Promega pGEM-T vector kit. The ligation mix was purified by isopropanol precipitation and the DNA resuspended in 15 μl water. 5 μl of each ligation mix was then used to transform DH5α competent cells by electroporation, and the cells and plated onto LB agar containing Ampicillin, IPTG and X-gal plates for blue and white colony screening. 15 white colonies were selected from each plate and grown in LB medium containing ampicillin, plasmid DNA prepared nucleotide sequences of the DNA inserts obtained by BigDye sequencing. FIG. 13 shows the products of the PCR amplification analysed by gel electrophoresis. FIGS. 14 to 18 show a comparison of the sequence isotypes found.

Analysis of the sequencing results using GCG (WebANGIS) showed different isotypes present in durum, oats and potato in the two isoamylase regions tested. As expected, the durum wheat sequences were highly similar to the A and B genomes of hexaploid wheat.

Molecular markers based on these polymorphisms are developed for these regions of the isoamylase genes to distinguish the different isotypes, as has been done for wheat.

### Example 8

### Linked Markers from Flanking Regions

Nucleotide sequence information from genome regions flanking the isoamylase genes can also be used to identify polymorphisms closely linked to the genes but outside of the genes themselves. Such polymorphisms can give rise to molecular markers that can be used to identify deletions in one genome of particular plant varieties, where the deletions extend to part of the isoamylase gene itself, thus identifying further null alleles. Polymorphisms outside of the genes are not represented in the isoamylase mRNA or cDNA sequences and therefore cannot be used to screen for mutants where the gene is present but expression is impaired.

Nucleotide sequences from flanking regions are obtained as follows. A library is made in a λ vector using total genomic DNA from the plant, and plaques obtained by plating 10⁶ or more λ infectious particles at high density in a suitable E. coli host. The plaques are transferred to nylon filters. An isoamylase gene-specific probe, such as those described above, is labelled with radioactive label and used to hybridise to the nylon filters. Plaques corresponding to spots of hybridisation are isolated and confirmed to be positive for isoamylase sequences by second or third rounds of hybridisation. DNA sequencing of the gene segments in the λ clones, particularly those flanking the isoamylase gene segments, is carried out by standard methods to determine the full nucleotide sequence of the inserts. The sequences of the isoamylase gene segments in each clone can be used to identify which genome the clone is derived from, and polymorphisms identified in the flanking regions identified accordingly. Alternatively, the euploid and chromosome engineered Chinese Spring lines can be used to determine the genome identity of the sequences flanking the isoamylase genes.

Any genetic polymorphism closely linked to the isoamylase gene can be used as a molecular marker, as exemplified in the Examples above.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein by reference in their entirety. Australian Provisional Application No. PR8827 filed 12 Nov. 2001 is incorporated herein by reference in its entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Ball S., Guan H. P., James M., Myers A., Keeling P., Mouille G., Buleon A., Colonna P., and Preiss J. (1996). From glycogen to amylopectin: a model for the biogenesis of the plant starch granule. Cell 86: 349-352.
Beatty M. K, Rahman A., Cao H., Woodman W., Lee M., Myers A. M., and James M. G. (1999). Purification and molecular genetic characterisation of ZPU, a pullulanase-type debranching enzyme from maize. Plant Physiology 119: 255-266.
Emanuelsson O., Nielsen H., and von Heijne G. (1999). ChloroP, a neural network-based method for predicting chloroplast transit peptides and their cleavage sites. Protein Science 8: 978-984.
Fujita N., Kubo A., Fransisco P. B., Nakakita M., Harada K, Minaka N., and Nakamura Y. (1999). Purification, characterisation and cDNA structure of isoamylase from developing endosperm of rice. Planta 208: 283-293.
Fukui K. N., Suzuki G., Lagudah E., Rahman S., Appels R., Yamamoto M., and Mukai Y. (2001). Physical arrangement of retrotransposon-related repeats in centromeric regions of wheat. Plant Cell Physiology 42: 189-196.
Genschel, U., Abel, G., Lorz, H., and Lutticke, S. (2002). The sugary-typeisoamylase in wheat: tissue distribution and subcellular localisation. Planta 214: 813-820.
James M. G., Robertson D. S., and Myers A. M. (1995). Characterisation of the maize gene sugary1, a determinant of starch composition in kernels. Plant Cell 7:417-429.
Jesperson H. M., MacGregor E. A., Henrissat B., Sierks M. R., and Svensson B. (1993). Starch and glycogen debranching and branching enzymes: Prediction of structural feautes of athe catalytic b/a barrel and evolutionary relationship to other amylolytic enzymes. Journal of Protein Chemistry 12: 791-805.
Kubo A., Fujita N., Harada K., Satoh H., and Nakamura Y. (1999). The starch debranching enzymes isoamylase and pullulanase are both involved in amylopectin biosynthesis in rice endosperm. Plant Physiology 121: 399-409.
Lagudah E. S., Appels R., and McNeil D. (1991). The Nor-D3 locus of Triticum tauschii: natural variation and genetic linkage to markers in chromosome 5. Genome 34: 387-395.
Langridge, P., Lagudah, E. S., Holton, T. A., Appels, R., Sharp, P. J. and Chalmers, K J. (2001). Trends in genetic and genome analysis in wheat: a review, Aust. J. Agric. Res. 52: 1043-1077.
Lemieux, B. (2000). High Throughput Single Nucleotide Polymorphism Genotyping Technology.
Current Genomics. 1: 301-311.
Li Z., Rahman S., Kosar-Hashemi B., Mouille G., Appels R., and Morell M. K (1999). Cloning and characterisation of a gene encoding starch synthase I. Theoretical and Applied Genetics 98: 1208-1216.
Nakamura Y., Umemoto T., Ogata N., Kuboki Y. Yano M., and Sasaki T. (1996). Starch debranching enzyme (R-enzyme or pullulanase) from developing rice endosperm: purification, cDNA and chromosomal localization of the gene. Planta 199: 209-218.
Needleman, S. B. and Wunsch, C. D. (1970). A general method applicable to the search for similarities in the amino acid sequence of two proteins. Journal of Molecular Biology 48: 443-453.
Rahman S., Abrahams S. L., Mukai Y., Abbott D. C., Samuel M. S., Morell M. K., and Appels R. (1997). A complex arrangement of genes at a SBE I locus in wheat. Genome 40: 465-474.
Rahman S., Li Z., Abrahams S. L., Abbott D. C., Appels R., and Morell M. K. (1999). Characterisation of a gene encoding wheat endosperm starch branching enzyme-I. Theoretical and Applied Genetics 98: 156-163.
Rayburn A. L., and Gill B. S. (1985). Molecular identification of the D-genome chromosomes of wheat. Journal of Heredity 77: 253-255.
Sears E. R., and Miller T. E. (1985). The history of Chinese Spring wheat. Cereal Research Communication 13: 261-263.
Thorbjornsen T., Villand P., Denyer K, O-A O., and Smith A. M. (1996). Distinct forms of ADPglucose pyrophosphorylase occur inside and outside and the amyloplasts in barley endosperm. Plant Journal 10: 243-250.
Turner M., Mukai Y., Leroy P., Charef B., Appels. R., and Rahman S. (1999). The Ha locus of wheat: identification of a polymorphic region for tracing grain hardness in crosses. Genome 42:1-9.

1. A method of identifying a polyploid plant which has at least one impaired isoamylase gene, the method comprising screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

2. The method of claim 1, wherein the impaired isoamylase gene does not express a functional isoamylase protein.

3. The method of claim 1, the method comprises amplifying a region of an isoamylase gene.

4. The method of claim 3, wherein an amplicon of different size is produced from each genome of the plant.

5. The method of claim 3, wherein the method further comprises cleaving the amplicon(s), and wherein the products of the cleavage are different sizes for each of the isoamylase genes of the plant.

6. The method of claim 5, wherein the cleaving step is performed by a restriction enzyme.

7. The method according to claim 3, wherein the lack of an amplified product indicates that a given genome of the plant does not express a functional isoamylase protein.

8. The method according to claim 3, wherein the amplification is performed using the polymerase chain reaction.

9. The method according to claim 3, wherein the amplification is performed in the presence of an oligonucleotide primer comprising a sequence selected from: GGGGGTAAATCTTTTGGTCAGC (SEQ ID NO: 1) or CTATTCTGGCTGTGGGAATACC (SEQ ID NO: 67) and/or CCTGGAACCTCTGGTCATTATG (SEQ ID NO:2).

10. The method of claim 1, the method further comprising contacting the plant, or protein containing material isolated from the plant, with an antibody that binds to at least one isoform, but not all isoforms, of an isoamylase from a polyploid plant, and determining the presence or absence of antibody bound protein.

11. The method of claim 10, wherein the lack of antibody binding indicates that at least one genome of the plant does not express a functional isoamylase.

12. The method of claim 10, wherein the antibody binds to the isoamylase protein at a region comprising a sequence selected from the group consisting of: TKAEDEDDDEEEAVA (SEQ ID NO:3), TKVEDEGEEDEPVA (SEQ ID NO:4), TLADLVTYNKKYN (SEQ ID NO:5), TLGDLVTYNNKYN (SEQ ID NO:6), FSPMPRSTSGG (SEQ ID NO:7), FSPMTRYTSGG (SEQ ID NO:8), GPILSFKGVD (SEQ ID NO:9), GPILSFRGVD (SEQ ID NO:10), VTEEVPLDPL (SEQ ID NO:1 1), VTEEVSLDPL (SEQ ID NO:12), FIEGELHNML (SEQ ID NO:13), FIEGELHDML (SEQ ID NO:14), PHCGHYLDVS (SEQ ID NO:15), PHCGHYLDIS (SEQ ID NO:16), WVTEMHVDGF (SEQ ID NO:17), and WVMEMHVDGF (SEQ ID NO:18).

13. The method according to claim 10, wherein the antibody is detectably labeled.

14. The method according to claim 1, wherein the plant comprises a polymorphism or mutation in a region between introns 7 and 9, or a region between exons 1 and 3, of the isoamylase gene when compared to a wildtype sequence.

15. The method of claim 14, wherein the region between introns 7 and 9 corresponds to between nucleotides 4681 and 5250 of SEQ ID NO:19.

16. The method according to claim 1, wherein the polyploid plant is selected from the group consisting of: wheat, oats, potato, cotton, cherry, sugar-cane and bananas.

17. The method of claim 16, wherein the polyploid plant is wheat.

18. A method of producing and identifying a polyploid plant which has at least one impaired isoamylase gene, the method comprising; i) exposing a polyploid plant, or a progenitor thereof, to conditions which promote DNA mutagenesis, and ii) screening the plants obtained from step i), or progeny thereof, to identify plants which have at least one impaired isoamylase gene.

19. The method of claim 18, wherein the screening of step ii) comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

20. A method of producing and identifying a polyploid plant which has at least two impaired isoamylase genes, the method comprising;
i) identifying a polyploid plant which has at least one impaired isoamylase gene using the method according to claim 1,
ii) exposing the plant, or a progenitor thereof, of step i) to conditions which promote DNA mutagenesis, and
iii) screening the plants obtained from step ii), or progeny thereof, to identify plants which have at least two impaired isoamylase genes.

21. The method of claim 20, wherein the screening of step iii) comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

22. A method of producing and identifying a polyploid plant which has at least two impaired isoamylase genes, the method comprising;
i) exposing a first polyploid plant, or a progenitor thereof, to conditions which promote DNA mutagenesis,
ii) screening the plants obtained from step i), or progeny thereof, to identify plants which have at least one impaired isoamylase gene,
iii) crossing the plant of step ii) which has at least one impaired isoamylase gene with a second polyploid plant which has at least one impaired isoamylase gene, wherein the first and second plants have at least one impaired isoamylase gene which is on a different genome when compared to the other plant, and
iv) screening the plants obtained from step iii), or progeny thereof, to identify plants which have at least two impaired isoamylase genes.

23. The method of claim 22, wherein the screening of step ii) comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

24. The method of claim 22, wherein the screening of step iv) comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

25. A method of producing a polyploid plant which has reduced levels of functional isoamylase protein, the method comprising;
i) crossing two parent polyploid plants each of which have at least one impaired isoamylase gene, and
ii) screening the plants obtained from step i), or progeny thereof, to identify plants which have at least two impaired isoamylase genes,
wherein each of the parent plants have at least one impaired isoamylase gene which is on a different genome when compared to the other parent plant.

26. The method of claim 25, wherein at least one of the parent plants used in the cross is identified by a method comprising screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

27. The method of claim 25, wherein the screening of step ii) comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

28. The method of claim 26, wherein at least one of the plants used in the cross comprises at least one impaired isoamylase gene that does so not express a functional isoamylase protein.

29. A method of introducing an impaired isoamylase gene into the genome of a polyploid plant, the method comprising;
i) crossing a first parent plant with a second parent plant, wherein the as second plant comprises at least one impaired isoamylase gene, and
ii) backcrossing the progeny of i) with plants of the same genotype as the first parent plant for a sufficient number of times to produce a plant with a majority of the genotype of the first parent but comprising the impaired isoamylase gene,
wherein progeny plants are screened for the impaired isoamylase gene.

30. The method of claim 29, wherein the screening comprises screening the plant for a polymorphism or mutation in an isoamylase gene which is linked to reduced production of a functional isoamylase protein, wherein wildtype plants accumulate starch in a storage organ of said wildtype plants, and wherein the corresponding isoamylase gene of said wildtype plants produce functional isoamylase in said storage organ.

31. A method of distinguishing between the A and B genomes of wheat, the method comprising detecting a nucleotide sequence difference between the isoamylase gene of the A and B genomes, wherein the plant expresses isoamylase in the endosperm of developing grain.

32. The method of claim 31, wherein the nucleotide sequence difference occurs in a region between introns 7 and 9, or a region between exons 1 and 3, of the isoamylase gene when compared to a wildtype sequence.

33. A plant produced by the method according to claim 18.

34. A non-transgenic polyploid plant comprising at least two impaired isoamylase genes, wherein wildtype plants of the same species of the polyploid plant accumulate starch in a storage organ of said wildtype plants, and wherein the corresponding isoamylase genes of said wildtype plants produce functional isoamylase in said storage organ.

35. The plant of claim 33 wherein the plant is a cereal.

36. The plant of claim 35, wherein the cereal is wheat.

37. The plant of claim 36, wherein the wheat comprises impaired isoamylase genes in the B and D genomes.

38. The plant of claim 36, wherein the wheat comprises impaired isoamylase 3 genes in the A and B genomes.

39. The plant of claim 36, wherein the wheat comprises impaired isoamylase genes in the A and D genomes.

40. The plant according to claim 36, wherein the plant is substantially lacking isoamylase activity in the endosperm during grain development.

41. A storage organ of the plant according to claim 33.

42. The storage organ of claim 41 which is a seed.

43. A food or non-food substance produced using a plant according to claim 30 or a storage organ thereof.

44. An antibody that binds to at least one isoform of an isoamylase from a polyploid plant.

45. The antibody of claim 44, wherein the antibody binds specifically to only one isoform of an isoamylase from a polyploid plant.

46. The antibody of claim 44, wherein the antibody binds specifically to all isoforms of isoamylase from a polyploid plant.

47. A substantially purified polypeptide comprising an amino acid sequence selected from the group consisting of:
i) a sequence provided as SEQ ID NO:20,
ii) a sequence provided as SEQ ID NO:21,
iii) a sequence provided as SEQ ID NO:22,
iv) a sequence that is at least 93% identical to any one of i) to iii) which is at least 535 amino acids in length,
v) a sequence provided as SEQ ID NO:23,
vi) a sequence provided as SEQ ID NO:24,
vii) a sequence provided as SEQ ID NO:25, and
viii) a sequence which is at least 93% identical to any one of v) to vii).

48. The polypeptide of claim 47 which has isoamylase activity.

49. The polypeptide of claim 47 which is substantially purified from wheat.

50. A fusion protein comprising a polypeptide according to claim 47 fused to at least one other polypeptide sequence.

51. An isolated polynucleotide comprising a nucleotide sequence selected from the group consisting of:
i) a sequence provided in SEQ ID NO: 50,
ii) a sequence provided in SEQ ID NO: 51,
iii) a sequence provided in SEQ ID NO: 52,
iv) a sequence encoding a polypeptide according to claim 47, and
v) a sequence which is at least 90% identical to any one of i) to iii) and which is at least 1710 nucleotides in length.

52. The polynucleotide of claim 51 which encodes a polypeptide which has isoamylase activity.

53. A vector comprising the polynucleotide claim 51.

54. The vector of claim 53 which is an expression vector.

55. A host cell comprising the vector of claim 53.

56. A plant transformed with a polynucleotide according to claim 51, wherein the polynucleotide is capable of being expressed in said plant.

57. A method of altering starch synthesis in a plant, the method comprising disrupting the production and/or activity of a polypeptide according to claim 47.

58. The method of claim 57, wherein starch synthesis is altered by as increasing the amount of free sugars, or by altering the proportions of amylose or amylopectin or phytoglycogen, in the plant.


SEQUENCE LISTING (67 sequences)

SEQ ID NO: 1 - DNA, 22 bp, Triticum aestivum
gggggtaaatcttttggtcagc

SEQ ID NO: 2 - DNA, 22 bp, Triticum aestivum
cctggaacctctggtcattatg

SEQ ID NO: 3 - protein, 15 aa, Triticum aestivum
TKAEDEDDDEEEAVA

SEQ ID NO: 4 - protein, 14 aa, Triticum aestivum
TKVEDEGEEDEPVA

SEQ ID NO: 5 - protein, 13 aa, Triticum aestivum
TLADLVTYNKKYN

SEQ ID NO: 6 - protein, 13 aa, Triticum aestivum
TLGDLVTYNNKYN

SEQ ID NO: 7 - protein, 11 aa, Triticum aestivum
FSPMPRSTSGG

SEQ ID NO: 8 - protein, 11 aa, Triticum aestivum
FSPMTRYTSGG

SEQ ID NO: 9 - protein, 10 aa, Triticum aestivum
GPILSFKGVD

SEQ ID NO: 10 - protein, 10 aa, Triticum aestivum
GPILSFRGVD

SEQ ID NO: 11 - protein, 10 aa, Triticum aestivum
VTEEVPLDPL

SEQ ID NO: 12 - protein, 10 aa, Triticum aestivum
VTEEVSLDPL

SEQ ID NO: 13 - protein, 10 aa, Triticum aestivum
FIEGELHNML

SEQ ID NO: 14 - protein, 10 aa, Triticum aestivum
FIEGELHDML

SEQ ID NO: 15 - protein, 10 aa, Triticum aestivum
PHCGHYLDVS

SEQ ID NO: 16 - protein, 10 aa, Triticum aestivum
PHCGHYLDIS

SEQ ID NO: 17 - protein, 10 aa, Triticum aestivum
WVTEMHVDGF

SEQ ID NO: 18 - protein, 10 aa, Triticum aestivum
WVMEMHVDGF

SEQ ID NO: 19 - DNA, 9643 bp, Aegilops tauschii
cgggaccgtcccttggcaacttgggttacgttgggacctgacgcttcgcttatccggtgtgccctgagacgagatatgtgcagctcctatcggatttgtcggcacattcggcggctttgctggtcttgttttaccattgtcgaaatgtcttataaaccgggattccgagactgatcgggtcttcccgggagaaggtttatccttcgttgaccgtgagagcttataatgggctaagttgggacacccctgcagggtattatctttcgaaagccgtgcccgcggttatgaggcagatgggaatttgttaatgtccgattgtagagaacctgtcacttgacttaatttaaaattcatcaaccgtgtgtgtagccgtgatggtctcttttcggcggagtccgggaagtgaacacggtttgagttatgcatgaacgtaagtagtttcaggatcactccttgatcacttctagctccgcgaccgttgcgttgtttctcttctcgctctcatttgcgtatgttagccaccatatatgcttagtgtctgctgcagctccacctcattaccccttcctttcctataagcttaaatagtcttgatctcgcgggtgtgagattgctgagtcctcgtgacttacagattctaccaaaacagttgcaggtgtcgacgatgccagtgcaggtgacgcaaccgagctcaagtgggagttcgacgaggaacgtggtcgttactatgtttcttttcctgatgatcagtagtggagcccagttgggacgatcggggatctagcatttggggttatcttaatttcttttagatttgaccgtaatcggtctatgtgtggattttggatgatgtatgaattatttatgtattgtgtgaagtggcgattgtaagccaactctcgttatcccattcttgttcattacatgggattgtgtgaagatgacccttcttgcgacaaaaccacaatgcggttatgcctctaagtcgtgcctcgacacgtgggagatatagccgcatcgtgggcgttacacgcaagtcttcatagcaaccaaaactcctctccgcattacaagccaccaatcgcagccaccatgactttcttcaccactgtcaatgccatgaaaatctatatgtagacatgtcccattgcatcggcaagaaagcgaagcttcacggcacaccttcatgaagcctctctggccgaagacaaggatgcgcccgaccggatcaattcctatctagatacctagtggagccatgcgccaatagcggagatctccgagaggaagaccggaactcgtcggacgtcggcgtccaaatcgaggaggccggcatgaagcacatcgaggatggtgatccccatacgggtagatcgggtcggccgccatctcacaccgagattaggatgcttaaaacggtttttttggcactagcattattttgcatcatccgttggagagaacatgagagagccccatttcttccacggttctacctatgggatcttgttctgcttgcaaccgggcctcacggaaaacccgcgccagcggacccaccccatgctagcagggcacggcacccgcagcggccggtccaaatggacggtgagaaccgcaacgcgacacgcccggcactgtcagcaaagcgagagcgcgcgcacggcacacgcacgctcggacgaacggacggtgcgatcgatccctcccccctcgctcaaccacagtagtaccctgccacactatcacgcacgcactcgagtcacacctcccacgaagaaccaacaggaggcgcggatcccaccgataaataaccccgcctcgccgctcctccccaaaatcaatcaccgatcgctcggggttcccggcatgacgatgatggccatggccaaggcgccctgcctctgcgcgcgcccgtccctcgccgcgcgcgcgaggcggccggggccggggccggcgccgcgcctgcgacggtggcgacccaatgcgacggcggggaagggggtcggcgaggtgtgcgccgcggttgtcgaggcggcgacgaaggccgaggatgaggacgacgacgaggaggaggcggtggcggaggacaggtacgcgctcggcggcgcgtgcagggtgctcgccggaatgcccgcgccgctgggcgccaccgcgctcgccggcggggtcaatttcgccgtctactccggtggagccaccgccgcggcgctctgcctcttcacgccagaagatctcaaggcggtggggttgcctcccgagtagagttcatcagctttgcgtgcgccgcgcgcccccttttctggcctgcgatttaagttttgtactgggggaaatgctgcaggatagggtgacggaggaggtttcccttgaccccctgatgaatcggactgggaacgtgtggcatgtcttcattgaaggcgagctgcacgacatgctttacgggtacaggttcgacggcacctttgctcctcactgcgggcactaccttgatatttccaatgtcgtggtggatccttatgctaaggtgatcatactttagctttacctgcatcttggtatttacagtagaaattgttacgtggacccttatttgttgccttttgtgttgctctaggcagtgataagccgaggggagtatggcgttccggcgcgtggtaacaattgctggcctcagatggctggcatgatccctcttccatatagcacggtatgcctgattgctgaaaatattggctgcatttgtttctctctttttctcatatttttctcctgtctttcacttgtactacattgcctcagacagtcatgatcaaagagagcagtgtcattagacatttgtagttgtctgctgactttgaccaaaacttgtaatttactgttgttaaaggtccttgaatcatatttttttataatattatgtttgcaagtggaagtaaagtgaaattgcatctagtatttgttgttgctgtcttagtcgtttaattggacatgcagtaaaaaggtttgcatctgcagtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggtacagctgtacttgctgactacataggataatttttaaagaaagctacatattagccagaatttgggttattacaaaaactactgcatactatagcagttacatgctcattatcgaggagatgctcacacgcatcttatttggatttaatacccaattctgtaatcatgactaaatatgttttcccagatctgcttatgaattccgtattatttgatattggactgttccctctacaggagcttggagttaattgtattgaattaatgccctgccatgagttcaacgagctggagtactcaacctcttcttccaagtaaggacatgaatttagtattagcctgccagcactgtttgagtgagagttcatacacattttgtgcctgcataactgatatttgttcaaactattttttttagcagtcactcaacagttttacatatatatataatatagactattcgtcaccctgggtgaggaatagttattcttcacccacctctattttaacatctatgcaccgtaattttacgtttcgtaaattttgtcttattttagagataaaaagagaacgtaagaaaacctataatcgtcgtaaaaaaaaatatgttacgtaaaattacaaatgtaaaaacatagtgtaaaatgtacataaaatacattttttgacctatattttttttgttaatgccaaattttatacagtaaatcaatatgaatgtaactatttgtatttcaaatgtaatttatttatgaaatggtcgtaagattacctcgggtgaagaataacttattctgcaccctgggtgatgaatagtaacactatatatatatatatatatatatatatatatatataccggctgctgctaatgatgttaatatttcgcaagtacctaagctggatttttctccatgagacatcaatccataattgaaattggtcacgacagttgaatagttgatagctgaaaatgaaatccagcatgctactgtcttgccatctccagacttgctaacatgaattttgtctgcctacctgtcatttgtaccaacgttcccaattgccctctcattattcgtgtgtaccatgcatatgtgttttaacatgattattgttggctatatttctctttggaaacatgactaatttatcacccgttttgtataaactgcttgttttcatatcaggatgaacttttggggatattctaccataaacttcttttcaccaatgacgagatacacatcaggcgggataaaaaactgtgggcgtgatgccataaatgagttcaaaacttttgtaagagaggctcacaaacggggaattgaggtaagcaagtcgtacgagttagttgctccttttgaacttatcaatttgatgcgaagacatgttactgctaggtgatcctggatgttgtcttcaaccatacagctgagggtaatgagaatggtccaatattatcatttaggggggtcgataatactacatactatatgcttgcacccaaggtgacagatctttcttgctgcgtaattgttctttcatagatgtatagagcatagatgtgttatgtagtagttctttttcaaggggattatgttcatgcagggagagttttataactattctggctgtgggaataccttcaactgtaatcatcctgtggttcgtcaattcattgtagattgtttaaggtacagatatacattttacttctagaactactttttcatttcttttgctgcttgtcattttgatatgattaatttgcaagcttgtgggggtaaatcttttggtcagcatattgtatctttaaatgtcacaaatactaatgtcctggtgcttattgatttggcatcttcaaattcttctccaatgaaaagggaaaaatctactgtatgtctcgtcaactaatttacttttgttttgcagatactgggtgatggaaatgcatgttgatggttttcgttttgatcttgcatccataatgaccagaggttccaggtaatttgtatttattgtttgtttgcgtgttgccttttcagaagattcttaaaagaatgtttcttttacaagtctgtgggatccagttaacgtgtatggagctccaatagaaggtgacatgatcacaacagggacacctcttgttactccaccacttattgacatgatcagcaatgacccaattcttggaggcgtcaaggtacttgtttcatccaacacctgttgtctgtgtgcattcaattgttttaatatggtaatgatcaatttcccaatgttgataaggaaaaaaaatgcaagtagctctctttatctgcttcttgtgagttatgctaaacatgtagatactactatatttcaactgtatatacttgacatattattgcttccttgggaggctctcttattcctttcccccgttgcaattatagctcattgctgaagcatgggatgcaggaggcctctatcaagtaggtcaattccctcactggaatgtttggtctgagtggaatgggaaggtaaggtacctgttaaaagtttgaatggcaaatactgatagaaatataacttatatttgcgacatatatagataaagcaaaataatacgcattccacctgaactttaaaggggcacgcagaattatcccgcatctgtctacaagaatgataacacatgtgctgaatagtgaagtactacttctcaaatgtctgaatgaacgcactaactcttgtgagtgtcaaccgagcaagaaatatttgagttttctgcaagaaattgttcatgttgtgctgtattatactccctccgtccgaaattatttgtcggagaaatggatgtatctagacgtattttagttctagatacatccatttttatccatttctgcaacaagtagttccggacggagggagtatcatttaacaaatatatgcaatgttcgaagtaaatccccacgaataagcatataagacgatattgctttttgacttgcaacacctaaacctcattgttttctcctaggattttgggtgttcgaagcaagcagctggtgatatttaatttacctttgcctttatttgtagcttgatttgagggtgcggcaaaggttttagcttagtagtgttttgtaaattattatagtttatgtatatactcctcatttgggcacttccgtactggtcccatagaagataaaaatggaatgatcacgaattctgggtgtctggccaataattgttgacaacactgttgcgcatttgatttttatcagggaatggaaaattgaaatcggtaagaaacattgcgatattaagcttgtatatgctaatgctggtggatctttaagagggaacatatgatctcgtgtgcatccatcttcaactaaaaaaatatgttgcacatctcccacgtcacttactagctatttcatccaagtactaacttgtgtggttgtctcctcagtaccgggacattgtgcgccaattcattaaaggcactgatggatttgctggtggttttgccgaatgtctttgtggaagtccacacctataccaggtaagttgtggcaatacttggaaatgggttgagtgaatgtcacatggattttttatatataccacatgatgatacacatgtaaatatataacgattatagtgtatgcatatgcatttggctaagaagtactccctcccttagtaaaagttagtacaaagttgagtcatctattttggaacggagggagtataagtgtatacactagtgcaatatataggttttaacacccaacttgccaatgaaggaacatagggctttctagttatcttatttatttgtctggtgaataatccactgaaaaattccagccatgtcattttttagggggggagaagaaactacattgatttttccccctaaaaaaagccatctcagatttcataggtaacttgcttttctgtaaagaaatgaaaacgacttcatactttctgtcgattataagtgtatacactagtgcaatatataggttttaacacccaacttgccaatgaaggaacatagggctttctagttatcttatttatttgtctggtgaataatccactgaaaaattccagccatgtcattttttagggggggagaagaaactatattgatttttccccctaaaaaaagccatctcagattcataggtaacttgcttttctgtaaagaaatgaaaacgacttcatactttctgtcggcgcttacttagctcgatggatatttgtaagatgaatgccaaatttaatttggcgggaaatttgatctgttattcacaaatttctatttggtttctctagcaatcaaaccagtaacttgttattggcactgcaatttcttattgattaatcaggcaggaggaaggaaaccttggcacagtatcaactttgtatgtgcacatgatggatttacactgggtgatttggtaacatataataacaagtacaatttaccaaatggggagaacaatagagatggagaaaatcacaatcttagctggaattgtggggaggtaattctgaactctcctttttttttgaaattttcatgctttacataatagtcaaatggctgacaaatgtcgttgtatggttctctctacctaaaccgttaaggcagtaagagtttccctacaagatctctttgttcgtataattgtattttctagagaaaagttgccttcaattttgtgcacgcggcagtacaggaattgtggttataaatattgatacaggctgaccatcgttactaatagggggaacaataagcacatttttttaatagcaaaggcatcacccttgttccgtttccaatgaaatcacagtatccgaaccataagttttacaagtatgcgtagagagaaataaagtatcaacccggcagaaacagttgtttcaggcgcaaagagaaaaggaaacgatatgctctattacatcaaccttttagcatttagggacgaccagcatcatcccatcttcaatcaactggagcgaggtcacctccaatcttctcagcagcctcagagtggtgacctcccaagcaagtgcatcagcatccatcatctgggggttgggcacataccatgagcacaatcacctgaatttgatgaattttcctctgtttaccttgcagcagacccctgccgtataaatggttttaaatgacagcatgttctttcagtttgagcaaaatttgtgcaattgcaaagaagctttagaatcatgtggaacatgcacttacatttcatctgaacaatataggaaggagaattcgcaagattgtctgtcaaaagattgaggaagaggcagatgcgcaatttctttgtttgtctcatggtttctcaagtaagacttatatctgatctcttcaatttttgagattgcctgtttttcacaatggcatatgttgtcaggtgaaacatccaatcccagtattaatagagccaacatgaagggattgcttatctgagatatctgccaaagttgaattcttagattcaccttcttcagtatttcagaccttctaagcattttcattttttttttcaattgttagggagttccaatgttttacatgggcgatgaatatggccacacaaaagggggcaacaacaatacatactgccatgattcttatgtcagtacaatttggtcacatattgttgttctaagtaactatcttcaaatctttgcattcatccgtcatggctcttctgtaggtcaattattttcgctgggataaaaaagaacaatactctgacttgcaaagattctgctgcctcatgaccaaattccgcaagtaagtattccgttgaataatttctgtgtagaaccactgaaggtgcctccaaacgctaagcgagcaaggtcaatttcacaccctaatcaagttggtgttgtctatttgtgtatttgatctgctgcactgtagggagtgcgagggtcttggccttgaggactttccaacggccgaacggctgcagtggcatggtcatcagcctgggaagcctgattggtctgagaatagccgattcgttgccttttccatggtacacatatagttctgacacttcactatagttgttttaaaaaagaaaatttaactcaaaagtaaattatggagagttttagaagtaacaaatatctttatgtatctcaccacagaaagatgaaagacagggcgagatctatgtggccttcaacaccagccacttaccggccgttgttgagctcccagagcgcgcagggcgccggtgggaaccggtggtggacacaggcaagccagcaccatatgacttcctcaccgacgacttacctgatcgcgctctcaccatacaccagttctctcatttcctcaactccaacctctaccccatgctcagctactcatcggtcatcctagtattgcgccctgatgtttgaagaacaaaaa

SEQ ID NO: 20 - protein, 791 aa, Triticum aestivum
MTMMAMAKAPCLCARPSLAARARRPGPGPAPRLRRWRPNATAGKGVGEVCAAVVEAATKAEDEDDDEEEAVAEDRYALGGACRVLAGMPAPLGATALAGGVNFAVYSGGATAAALCLFTPEDLKADRVTEEVSLDPLMNRTGNVWHVFIEGELHDMLYGYRFDGTFAPHCGHYLDISNVVVDPYAKAVISRGEYGVPARGNNCWPQMAGMIPLPYSTFDWEGDLPLRYPQKDLVIYEMHLRGFTKHDSSNVEHPGTFIGAVSKLDYLKELGVNCIELMPCHEFNELEYSTSSSKMNFWGYSTINFFSPMTRYTSGGIKNCGRDAINEFKTFVREAHKRGIEVILDVVFNHTAEGNENGPILSFRGVDNTTYYMLAPKGEFYNYSGCGNTFNCNHPVVRQFIVDCLRYWVMEMHVDGFRFDLASIMTRGSSLWDPVNVYGAPIEGDMITTGTPLVTPPLIDMISNDPILGGVKLIAEAWDAGGLYQVGQFPHWNVWSEWNGKYRDIVRQFIKGTDGFAGGFAECLCGSPHLYQAGGRKPWHSINFVCAHDGFTLGDLVTYNNKYNLPNGENNRDGENHNLSWNCGEEGEFARLSVKRLRKRQMRNFFVCLMVSQGVPMFYMGDEYGHTKGGNNNTYCHDSYVNYFRWDKKEQYSDLHRFCCLMTKFRKECDGLGLEDFPTAERLQWHGHQPGKPDWSENSRFVAFSMKDERQGEIYVAFNTSHLPAVVELPERAGRRWEPVVDTGKPAPYDFLTDDLPDRALTIHQFSHFLNSNLYPMLSYSSVILVLRPDV

SEQ ID NO: 21 - protein, 790 aa, Triticum aestivum
MTMMAMPEAPCLCARPSLAARARRPGSGPAPRLRRWRPNATAGKGVGEVCAAVVEAATKVEDEGEEDEPVAEDRYALGGACRVLAGMPAPLGATALAGGVNFAVYSGGATAAALCLFTPEDLKADRVTEEVPLDPLMNRTGNVWHVFIEGELHNMLYGYRFDGTFAPHCGHYLDVSNVVVDPYAKAVISRGEYGVPARGNNCWPQMAGMIPLPYSTFDWEGDLPLRYPQKDLVIYEMHLRGFTKHDSSNVEHPGTFIGAVSKLDYLKELGVNCIELMPCHEFNELEYSTSSSKMNFWGYSTINFFSPMTRYTSGGIKNCGRDAINEFKTFVREAHKRGIEVILDVVFNHTAEGNENGPILSFKGVDNTTYYMLAPKGEFYNYSGCGNTFNCNHPVVRQFIVDCLRYWVTEMHVDGFRFDLASIMTRGSSLWDPVNVYGAPIEGDMITTGTPLVTPPLIDMISNDPILGGVKLIAEAWDAGGLYQVGQFPHWNVWSEWNGKCRDIVRQFIKGTDGFAGGFAECLCGSPHLYQAGGRKPWHSINFVCAHDGFTLADLVTYNKKYNLPNGENNRDGENHNLSWNCGEEGEFARLSVKRLRKRQMRNFFVCLMVSQGVPMFYMGDEYGHTKGGNNNTYCHDSYVNYFRWDKKEQYSELHRFCCLMTKFRKECEGLGLEDFPTAKRLQWHGHQPGKPDWSENSRFVAFSMKDERQGEIYVAFNTSHLPAVVELPERAGRRWEPVVDTGKPAPYDFLTDDLPDRALTIHQFSHFLYSNLYPMLSYSSVILVLRPDV

SEQ ID NO: 22 - protein, 720 aa, Triticum aestivum
AEDRYALGGACRVLAGMPAPLGATALAGGVNFAVYSGGATAAALCLFTPEDLKADRVTEEVPLDPLMNRTGNVWHVFIEGELHNMLYGYRFDGTFAPHCGHYLDVSNVVVDPYAKAVISRGEYGVPARGNNCWPQMAGMIPLPYSTFDWEGDLPLRYPQKDLVIYEMHLRGFTKHDSSNVEHPGTFIGAVSKLDYLKELGVNCIELMPCHEFNELEYSTSSSKMNFWGYSTINFFSPMPRSTSGGIKNCGRDAINEFKTFVREAHKRGIEVILDVVFNHTAEGNENGPILSFKGVDNTTYYMLAPKGEFYNYSGCGNTFNCNHPVVRQFIVDCLRYWVTEMHVDGFRFDLASIMTRGSSLWDPVNVYGAPIEGDMITTGTPLVTPPLIDMISNDPILGGVKLIAEAWDAGGLYQVGQFPHWNVWSEWNGKYRDIVRQFIKGTDGFAGGFAECLCGSPHLYQAGGRKPWHSINFVCAHDGFTLADLVTYNKKYNLPNGENNRDGENHNLSWNCGEEGEFARLSVKRLRKRQMRNFFVCLMVSQGVPMFYMGDEYGHTKGGNNNTYCHDSYVNYFRWDKKEQYSELHRFCCLMTKFRKECEGLGLEDFPTAKRLQWHGHQPGKPDWSENSRFVAFSMKDERQGEIYVAFNTSHLPAVVELPERAGRRWEPVVDTGKPAPYDFLTDDLPDRALTIHQFSHFLYSNLYPMLSYSSVILVLRPDV

SEQ ID NO: 23 - protein, 259 aa, Triticum aestivum
MTMMAMAKAPCLCARPSLAARARRPGPGPAPRLRRWRPNATAGKGVGEVCAAVVEAATKAEDEDDDEEEAVAEDRYALGGACRVLAGMPAPLGATALAGGVNFAVYSGGATAAALCLFTPEDLKADRVTEEVSLDPLMNRTGNVWHVFIEGELHDMLYGYRFDGTFAPHCGHYLDISNVVVDPYAKAVISRGEYGVPARGNNCWPQMAGMIPLPYSTFDWEGDLPLRYPQKDLVIYEMHLRGFTKHDSSNVEHPGTFIG

SEQ ID NO: 24 - protein, 258 aa, Triticum aestivum
MTMMAMPEAPCLCARPSLAARARRPGSGPAPRLRRWRPNATAGKGVGEVCAAVVEAATKVEDEGEEDEPVAEDRYALGGACRVLAGMPAPLGATALAGGVNFAVYSGGATAAALCLFTPEDLKADRVTEEVPLDPLMNRTGNVWHVFIEGELHNMLYGYRFDGTFAPHCGHYLDVSNVVVDPYAKAVISRGEYGVPARGNNCWPQMAGMIPLPYSTFDWEGDLPLRYPQKDLVIYEMHLRGFTKHDSSNVEHPGTFIG

SEQ ID NO: 25 - protein, 188 aa, Triticum aestivum
AEDRYALGGACRVLAGMPAPLGATALAGGVNFAVYSGGATAAALCLFTPEDLKADRVTEEVPLDPLMNRTGNVWHVFIEGELHNMLYGYRFDGTFAPHCGHYLDVSNVVVDPYAKAVISRGEYGVPARGNNCWPQMAGMIPLPYSTFDWEGDLPLRYPQKDLVIYEMHLRGFTKHDSSNVEHPGTFIG

SEQ ID NO: 26 - DNA, 306 bp, Artificial Sequence
  Consensus Sequence
aactgtaatcatcctgtggttcgtcaattcattgtagattgtttaaggtacagatatacattttacttctagaactacttttcatttctttcgctgcttgtcattttgatatgattaatttgcaagcttgtgggggtaaatcttttggtcagcatattgtatctttaaatgtcacaatactaatgtcctggtgcttattgatttggcatcttcaaattctttttctccaatgaaaagggaaaaatctactgtatgtctcgtcaactaatttacttttgttttgcagatactgggtgatggaaatga

SEQ ID NO: 27 - DNA, 300 bp, Triticum aestivum
  misc_feature (20)..(20): n = A, C, G or T
aactgtaatcatcctgtggntcgtcaattcattgtagattgtttaaggtacagatatacattttacttctagaactacttttcatttctttcgctgcttgtcattttgatatgattaatttgcaagcttgtggggtaaatcttttggtcagcatattgtatctttaaatgtcacctaatgtcttggtgcttatagatttggcatcttcaaattctttttctccaatgaaaagggaaaaatctactgtatgtctggtcaactaatttacttttgttttgcagatctgggtgatggaaatga

SEQ ID NO: 28 - DNA, 307 bp, Triticum aestivum
aactgtaatcatcctgtggttcgtcaattcattgtagattgtttaaggtacagatatacattttacttctagaactacttttcatttctttcgctgcttgtcattttgatatgattaatttgcaagcttgtgggggtaaatcttttggtcagcatattgtatctttaaatgtcaccaatactaatgtcctggtgcttattgatttggcatcttcaaattctttttctccagtgaaaagggaaaaatctactgtatgtctcgtcaactaatttacttttattttgcagatactgggtgacggaaatgc

SEQ ID NO: 29 - DNA, 303 bp, Triticum aestivum
aactgtaatcatcctgtggttcgtcaattcattgtagattgtttaaggtacagatatacattttacttctagaactactttttcatttcttttgctgcttgtcattttgatatgattaatttgcaagcttgtgggggtaaatcttttggtcagcatattgtatctttaaatgtcacaaatactaatgtcctggtgcttattgatttggcatcttcaaattcttctccaatgaaaagggaaaaatctactgtatgtctcgcaactaattacttttgttttgcagatactgggtgatggaaatga

SEQ ID NO: 30 - DNA, 494 bp, Avena sativa
cttcacgccagaagatctcaaggcggtgggcttgcttcccgagtagaattcaccattgcgtgtgctccttcttttagtttgcggtttgagttttctcttgcgggcaatggtgcagaatagggtcaccgaggaggttctcctcgatcccttgatcaacaggacggggaacgtgtggcatgttttcgtagaaggcgaggttcacaacatgctctacgggtacaggttcgatggcacctttgcccctgagtgcgggcagtacctcgatgcttccaacatcgtggtggatccttatgctaaggtgattgcgcgttagctttgctttgcctggtgatgtttacagcagaaattgtatgtactccgtagtaggtacttacttgctccgcctttttgtgttgctccaggcagtgataagccgaggggagtatggtgttccggggcctggtggggattgctggcctcaaatggctggcatgatccctcttccatatagcacg

SEQ ID NO: 31 - DNA, 494 bp, Avena sativa
cttcacgccagaagatctcaaggcggtgggcttgcttcccgagtagaattcaccattgcgtgtgctccttcttttagtctgcggtttgagttttctcttgcgggcaatggtgcagaatagggtcaccgaggaggttctcctcgatcccttgatcaacaggacggggaacgtgtggcatgttttcgtagaaggcgaggttcacaacatgctctacgggtacaggttcgatggcacctttgcccctgagtgcgggcagtacctcgatgcttccaacatcgtggtggatccttatgctaaggtgattgcgcgttagctttgctttgcctggtgatgtttacagcagaaattgtatgtactccgtagtaggtacttacttgctccgcctttttgtgttgctccaggcagtgataagccgaggggagtatggtgttccggggcctggtggggattgctggcctcaaatggctggcatgatccctcttccatatagcacg

SEQ ID NO: 32 - DNA, 494 bp, Avena sativa
cttcacgccagaagatctcaaggcggtgggcttgcttcccgagtagaattcaccattgcgtgtgctccttcttttagtctgcggtttgagttttctcttgcgggcaatggtgcagagtagggtcaccgaggaggttctcctcgatcccttgatcaacaggacggggaacgtgtggcatgttttcgtagaaggcgaggttcacaacatgctctacgggtacaggttcgatggcacctttgcccctgagtgcgggcagtacctcgatgcttccaacatcgtggtggatccttatgctaaggtgattgcgcgttagctttgctttgcctggtgatgtttacagcagaaattgtatgtactccgtagtaggtacttacttgctccgcctttttgtgttgctccaggcagtgataagccgaggggagtatggtgttccggggcctggtggggattgctggcctcaaatggctggcatgatccctcttccatatagcacg

SEQ ID NO: 33 - DNA, 494 bp, Avena sativa
cttcacgccagaagatctcaaggcggtgggcttgcttcccgagtagaattcaccattgcgtgtgctccttcttttagtctgcggtttgagttttctcttgcgggcaatggtgcagaatagggtcaccgagggggttctcctcgatcccttgatcaacaggacggggaacgtgtggcatgttttcgtagaaggcgaggttcacaacatgctctacgggtacaggttcgatggcacctttgcccctgagtgcgggcagtacctcgatgcttccaacatcgtggtggatccttatgctaaggtgattgcgcgttagctttgctttgcctggtgatgtttacagcagaaattgtatgtactccgtagtaggtacttacttgctccgcctttttgtgttgctccaggcagtgataagccgaggggagtatggtgttccggggcctggtggggattgctggcctcaaatggctggcatgatccctcttccatatagcacg

SEQ ID NO: 34 - DNA, 494 bp, Avena sativa
cttcacgccagaagatctcaaggcggtgggcttgcttcccgagtagaattcaccattgcgtgtgctccttcttttagtctgcggtttgagttttctcttgcgggcaatggtgcagaatagggtcaccgaggaggttctcctcgatcccttgatcaacaggacggggaacgtgtggcatgttttcgtagaaggcgaggttcacaacatgctctacgggtacaggttcgatggcacctttgcccctgagtgcgggcagtacctcgatgcttccaacatcgtggtggatccttatgctaaggtgattgcgcgttagctttgctttgcctggtgatgtttacagcaaaaattgtatgtactccgtagtaggtacttacttgctccgcctttttgtgttgctcctggcagtgataagccgaggggagtatggtgttccggggcctggtggggattgctggcctcaaatggctggcatgatccctcttccatatagcacg

SEQ ID NO: 35 - DNA, 490 bp, Triticum turgidum
cttcacgccagaagatctcaaggcggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgcggacgcttatttgctgccttttgtgttgctctaggcagtgataagccgaggggagtatggtgttccggcgcgtggtaacaattgctggcctcagatggctggcatgatccctcttccatatagcacg

SEQ ID NO: 36 - DNA, 491 bp, Triticum turgidum
cttcacgccagaagatctcaaggcggtggggttgcctcccgagtagagttcatcagctttgcgtgcgccgcgcgccccttttttgggcctgcaatttaagttttgtactggggcaaatgctgcaggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgaggacgcttatttgctgccttttgtgttgctccaggcagtgataagccgaggggagtatggtgttccagcgcgtggtaacaattgctggcctcagatggctggcatgatccctcttccatatagcacg

SEQ ID NO: 37 - DNA, 490 bp, Solanum tuberosum
cttcacgccagaagatctcaaggcggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattaactttacctgcatcttggtatttacagtagaaattgttacgcggacgcttatttgctgccttttgtgttgctctaggcagtgataagccgaggggagtatggtgttccggcgcgtggtaacaattgctggcctcagatggctggcatgatccctcttccatatagcacg

SEQ ID NO: 38 - DNA, 490 bp, Solanum tuberosum
cttcacgccagaagatctcaaggcggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgcttccaatgtcgtggtggatccttatgctaaggtgatcatacattagctttacctgcatcttggtatttacagtagaaattgttacgcggacgcttatttgctgccttttgtgttgctctaggcagtgataagccgaggggagtatggtgttccggcgcgtggtaacaattgctggcctcagatggctggcatgatccctcttccatatagcacg

SEQ ID NO: 39 - DNA, 490 bp, Solanum tuberosum
cttcacgccagaagatctcaaggcggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgcggacgcttatttgctgccttttgtgttgctctaggcagtgataagccgaggggagtatggtgttccggcgcgtggtaacaattgctgacctcagatggctggcatgatccctcttccatatagcacg

SEQ ID NO: 40 - DNA, 490 bp, Solanum tuberosum
cttcacgccagaagatctcaaggcggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgcggacgcttatttgctgccttttgcgttgctctaggcagtgataagccgaagggagtatggtgttccggcgcgtggtaacaattgctggcctcagatggctggcatgatccctcttccatatagcacg

SEQ ID NO: 41 - DNA, 490 bp, Solanum tuberosum
cttcacgccagaagatctcaagacggtggggttgcctcccgagtagagttcatcagctttgcttgcgccgcccgccccctttttggcctgcgatttgagttttgtatcgggggaaatgctgcaggatagggtgacggaggaggttccccttgaccccctgatgaatcggactgggagcgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgcggacgcttatttgctgccttttgtgttgctctaggcagtgataagccgaggggagtatggtgttccggcgcgtggtaacaattgctggcctcagatggctggcacgatccctcttccatatagcacg

SEQ ID NO: 42 - DNA, 491 bp, Solanum tuberosum
cttcacgccagaagatctcaaggcggtggggttgcctcccgagtagagttcatcagctttacgtgcgccgcgcgccccttttttgggcctgcaatttaagttttgtactggggcaaatgctgcaggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgaggacgcttatttgctgccttttgtgttgctccaggcagtgataagccgaggggagtatggtgttccagcgcgtggtaacaattgctggcctcagatggctggcatgatccctcttccatatagcacg

SEQ ID NO: 43 - DNA, 478 bp, Avena sativa
tctattctggctgtgggaataccttcaactgtaatcaccctgttgtccgtgaattcattgtagattgtttgaggtacagatgtgagtgtaactttcagaaagtaattttcagttttgtttcactgcttgtcattttaatatgattaagtcgcaagcttggaatgaatcttctgatcagcatattggcatcttgaaactcttctttggtaaaagagaaatccattgcatcttcacatcaatttttggatgtttatgaataaacagaactagtatgttgcttgggaaacgcttgaatcttctgttgacccatcttcatcatatatttgatgcaagcatatttgcatttattctataaacttgtcaagacaaacaatttatccagtactaattaagtttgctttgtagatactgggtgactgaaatgcatgttgatggttttcgttttgatcttgcatccataatgaccagaggttccagg

SEQ ID NO: 44 - DNA, 478 bp, Avena sativa
tctattctggctgtgggaataccttcaactgtaatcaccctgttgtccgtgaattcattgtagattgtttgaggtacagatatgagtgtaactttcagaaagtaattttcagttttgtttcactgcttgtcattttaatatgattaagtcgcaagcttggaatgaatcttctgatcagcatattggcatcttgaaactcttctttggtaaaagagaaatccattgcatcttcacatcaatttttggatgtttatgaataaacagaactagtatgttgcttgggaaacgcttgaatcttctgttgacccatcttcatcatatatttgatgcaagcatatttgcatttattctataaacttgtcaagacaaacaatttatccagtactaattaagtttgctttgtagatactgggtgactgaaatgcatgttgatggttttcgttttgatcttgcatccataatgaccagaggttccagg

SEQ ID NO: 45 - DNA, 478 bp, Avena sativa
tctattctggctgtgggaataccttcaactgtaatcaccctgttgtccgtgaattcattgtagattgtttgaggtacagatatgagtgtaactttcagaaagtaattttcagttttgtttcactgcttgtcattttaatatgattaagtcgcaagcttggaatgaatcttctgatcagcatattggcatcctgaaactcttctttggtaaaagagaaatccattgcatcttcacatcaatttttggatgtttatgaataaacagaactagtatgttgcttgggaaacgcttgaatcttctgttgacccatcttcatcatatatttgatgcaagcatatttgcatttattctataaacttgtcaagacaaacaatttatccagtactaattaagtttgctttgtagatactgggtgactgaaatgcatgttgatggttttcgttttgatcttgcatccataatgaccagaggttccagg

SEQ ID NO: 46 - DNA, 478 bp, Avena sativa
tctattctggctgtgggaataccttcaactgtaatcaccctgttgtccgtgaattcattgtagattgtttgaggtacagatatgagtgtaactttcagaaagtaattttcagttttgtttcactgcttgtcattttaatatgattaagtcgcaagcttggaatgaatcttcggatcagcatattggcatcttgaaactcttctttggtaaaagagaaatccattgcatcttcacatcaatttttggatgtttatgaataaacagaactagtatgttgcttgggaaacgcttgaatcttctgttgacccatcttcatcatatatttgatgcaagcatatttgcatttattctataaacttgtcaagacaaacaatttatccagtactaattaagtttgctttgtagatactgggtgactgaaatgcatgttgatggttttcgttttgatcttgcatccataatgaccagaggttccagg

SEQ ID NO: 47 - DNA, 530 bp, Avena sativa
tctattctggctgtgggaataccttcaactgtaatcaccctgtcgtccgtgaattcattgtagattgtttgaggtacagatataagtgtaacttccagaaagtaatttttagttttgtttcactgcttgtcattttaatatgattaagtcgcaagcttgtgaggatgaattttctgatcagcatattgtatctttaaatgttattacaaacactaattttttggtgcttatagatttggcgtcttcaaactcttcttcggtgaaagggaactctattgtatattcccatcaaatgtttggatgtttatgaataaaccaaactaatatatttcttgggaaaaacttgaatcttctgttgaaccatcttcatcatatatttgagcaagcatattttcatttattctatatacttttcaagacaaactatttattcagtactaattaagtttctttgtagatactgggtgactgaaatgcatgttgatggttttcgttttgatcttgcatccataatgaccagaggttccagg

SEQ ID NO: 48 - DNA, 529 bp, Avena sativa
tctattctggctgtgggaataccttcaactgtaatcaccctgtcgtccgtgaattcattgtagattgtttgaggtacggatataagtgtaacttccagaaagtaatttttagttttgtttcactgcttgtcatttaatatgattaagtcgcaagcttgtgaggatgaattttctgatcagcatattgtatctttaaatgttattacaaacactaattttttggtgcttatagatttggcgtcttcaaactcttcttcggtgaaagagaactctattgtatattcccatcaaatgtttggatgtttatgaataaaccaaactaatatatttcttgggaaaaacttgaatcttctgttgaaccatcttcatcatatatttgagcaagcatattttcatttattctatatacttttcaagacaaactatttattcagtactaattaagtttctttgtagatactgggtgactgaaatgcatgttgatggttttcgttttgatcttgcatccataatgaccagaggttccagg

SEQ ID NO: 49 - DNA, 379 bp, Triticum turgidum
ctattctggctgtgggaataccttcaactgtaatcatcctgtggttcgtcaattcattgtagattgtttaaggtacagatatacattttacttctagaactacttttcatttctttcgctgcttgtcattttgatatgattaatttgcaagcttgtggggtaaatcttttggtcagcatattgtatctttaaatgtcacctaatgtcttggtgcttatagatttggcatcttcaaattctttttctccaatgaaaagggaaaaatctactgtatgtctggtcaaccaatttacttttgttttgcagatactgggtgacggaaatgcatgttgatggttttcgttttgatcttgcatccataatgaccagaggttccagg

SEQ ID NO: 50 - DNA, 2373 bp, Triticum aestivum
atgacgatgatggccatggccaaggcgccctgcctctgcgcgcgcccgtccctcgccgcgcgcgcgaggcggccggggccggggccggcgccgcgcctgcgacggtggcgacccaatgcgacggcggggaagggggtcggcgaggtgtgcgccgcggttgtcgaggcggcgacgaaggccgaggatgaggacgacgacgaggaggaggcggtggcggaggacaggtacgcgctcggcggcgcgtgcagggtgctcgccggaatgcccgcgccgctgggcgccaccgcgctcgccggcggggtcaatttcgccgtctactccggtggagccaccgccgcggcgctctgcctcttcacgccagaagatctcaaggcggatagggtgacggaggaggtttcccttgaccccctgatgaatcggactgggaacgtgtggcatgtcttcattgaaggcgagctgcacgacatgctttacgggtacaggttcgacggcacctttgctcctcactgcgggcactaccttgatatttccaatgtcgtggtggatccttatgctaaggcagtgataagccgaggggagtatggcgttccggcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaagatgaacttttggggatattctaccataaacttcttttcaccaatgacgagatacacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaggggggtcgataatactacatactatatgcttgcacccaagggagagttttataactattctggctgtgggaataccttcaactgtaatcatcctgtggttcgtcaattcattgtagattgtttaagatactgggtgatggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtaccgggacattgtgcgccaattcattaaaggcactgatggatttgctggtggttttgccgaatgtctttgtggaagtccacacctataccaggcaggaggaaggaaaccttggcacagtatcaactttgtatgtgcacatgatggatttacactgggtgatttggtaacatataataacaagtacaatttaccaaatggggagaacaatagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttttacatgggcgatgaatatggccacacaaaagggggcaacaacaatacatactgccatgattcttatgtcaattattttcgctgggataaaaaagaacaatactctgacttgcacagattctgctgcctcatgaccaaattccgcaaggagtgcgatggtcttggccttgaggactttccaacggccgaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatatgacttcctcaccgacgacttacctgatcgcgctctcaccatacaccagttctctcatttcctcaactccaacctctaccccatgctcagctactcatcggtcatcctagtattgcgccctgatgtt

SEQ ID NO: 51 - DNA, 2370 bp, Triticum aestivum
atgacaatgatggccatgcccgaggcgccctgcctctgcgcgcgcccgtccctcgccgcgcgcgcgaggcggccggggtcggggccggcgccgcgcctgcgacggtggcgacccaatgcgacggcggggaagggggtcggcgaggtgtgcgccgcggttgtcgaggcggcgacgaaggtagaggacgagggggaggaggacgagccggtggcggaggacaggtacgcgctcggcggcgcgtgcagggtgctcgccggaatgcccgcgccgctgggcgccaccgcgctcgccggcggggtcaatttcgccgtctattccggcggagccaccgccgcggcgctctgcctcttcacgccagaagatctcaaggcggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggcagtgataagccgaggggagtatggtgttccagcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaagatgaacttttggggatattctaccataaacttcttttcaccaatgacaagatacacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaagggggtcgataatactacatactatatgcttgcacccaagggagagttttataactattctggctgtgggaataccttcaactgtaatcatcctgtggttcgtcaattcattgtagattgtttaagatactgggtgacggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtgccgggacattgtgcgtcaattcattaaaggcactgatggatttgctggtggttttgccgaatgtctttgtggaagtccacacctataccaggcaggaggaaggaaaccttggcacagtatcaactttgtatgtgcacatgatggatttacactggctgatttggtaacatataataagaagtacaatttaccaaatggggagaacaacagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttctacatgggtgatgaatatggccacacaaaagggggcaacaacaatacatactgccatgattcttatgtcaattattttcgctgggataaaaaagaacaatactctgagttgcaccgattctgctgcctcatgaccaaattccgcaaggagtgcgagggtcttggccttgaggactttccaacggccaaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatacgacttcctcaccgacgacttacctgatcgcgctctcaccatacaccagttctcgcatttcctctactccaacctctaccccatgctcagctactcatcggtcatcctagtattgcgccctgatgtt

SEQ ID NO: 52 - DNA, 2160 bp, Triticum aestivum
gcggaggacaggtacgcgctcggcggcgcgtgcagggtgctcgccggaatgcccgcgccgctgggcgccaccgcgctcgccggcggggtcaatttcgccgtctattccggcggagccaccgccgcggcgctctgcctcttcacgccagaagatctcaaggcggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggcagtgataagccgaggggagtatggtgttccagcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaaaatgaacttttggggatattctaccataaacttcttttcaccaatgccaagatccacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaagggggtcgataatactacatactatatgcttgcacccaagggagagttttataactattctggctgtgggaataccttcaactgtaatcatcctgtggttcgtcaattcattgtagattgtttaagatactgggtgacggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtaccgggacattgtgcgtcaattcattaaaggcactgatggatttgctggtggttttgccgaatgtctttgtggaagtccacacctataccaggcaggaggaaggaaaccttggcacagtatcaactttgtatgtgcacatgatggatttacactggctgatttggtaacatataataagaagtacaatttaccaaatggggagaacaacagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttctacatgggtgatgaatatggccacacaaaagggggcaacaacaatacatactgccatgattcttatgtcaattattttcgctgggataaaaaagaacaatactctgagttgcaccgattctgctgcctcatgaccaaattccgcaaggagtgcgagggtcttggccttgaggactttccaacggccaaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatacgacttcctcaccgacgacttacctgatcgcgctctcaccatacaccagttctcgcatttcctctactccaacctctaccccatgctcagctactcatcggtcatcctagtattgcgccctgatgtt

SEQ ID NO: 53 - DNA, 2550 bp, Triticum aestivum
gttcccggcatgacaatgatggccatgcccgaggcgccctgcctctgcgcgcgcccgtccctcgccgcgcgcgcgaggcggccggggtcggggccggcgccgcgcctgcgacggtggcgacccaatgcgacggcggggaagggggtcggcgaggtgtgcgccgcggttgtcgaggcggcgacgaaggtagaggacgagggggaggaggacgagccggtggcggaggacaggtacgcgctcggcggcgcgtgcagggtgctcgccggaatgcccgcgccgctgggcgccaccgcgctcgccggcggggtcaatttcgccgtctattccggcggagccaccgccgcggcgctctgcctcttcacgccagaagatctcaaggcggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggcagtgataagccgaggggagtatggtgttccagcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaagatgaacttttggggatattctaccataaacttcttttcaccaatgacaagatacacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaagggggtcgataatactacatactatatgcttgcacccaagggagagttttataactattctggctgtgggaataccttcaactgtaatcatcctgtggttcgtcaattcattgtagattgtttaagatactgggtgacggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtgccgggacattgtgcgtcaattcattaaaggcactgatggatttgctggtggttttgccgaatgtctttgtggaagtccacacctataccaggcaggaggaaggaaaccttggcacagtatcaactttgtatgtgcacatgatggatttacactggctgatttggtaacatataataagaagtacaatttaccaaatggggagaacaacagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttctacatgggtgatgaatatggccacacaaaagggggcaacaacaatacatactgccatgattcttatgtcaattattttcgctgggataaaaaagaacaatactctgagttgcaccgattctgctgcctcatgaccaaattccgcaaggagtgcgagggtcttggccttgaggactttccaacggccaaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatacgacttcctcaccgacgacttacctgatcgcgctctcaccatacaccagttctcgcatttcctctactccaacctctaccccatgctcagctactcatcggtcatcctagtattgcgccctgatgtttgagagaccaatatatacagtaaataatatgtctatatgtagtcctttggcgtattatcagtgtgcacaattgctctattgccagtgatctattcgatccacagatacatgtgcaaactgcaaagtttctggtaatcagagaagtttttcctgacgaccaagctactatac

SEQ ID NO: 54 - DNA, 2314 bp, Triticum aestivum
ggcggaggacaggtacgcgctcggcggcgcgtgcagggtgctcgccggaatgcccgcgccgctgggcgccaccgcgctcgccggcggggtcaatttcgccgtctattccggcggagccaccgccgcggcgctctgcctcttcacgccagaagatctcaaggcggatagggtgaccgaggaggttccccttgaccccctgatgaatcggaccgggaacgtgtggcatgtcttcatcgaaggcgagctgcacaacatgctttacgggtacaggttcgacggcacctttgctcctcactgcgggcactaccttgatgtttccaatgtcgtggtggatccttatgctaaggcagtgataagccgaggggagtatggtgttccagcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaaaatgaacttttggggatattctaccataaacttcttttcaccaatgccaagatccacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaagggggtcgataatactacatactatatgcttgcacccaagggagagttttataactattctggctgtgggaataccttcaactgtaatcatcctgtggttcgtcaattcattgtagattgtttaagatactgggtgacggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtaccgggacattgtgcgtcaattcattaaaggcactgatggatttgctggtggttttgccgaatgtctttgtggaagtccacacctataccaggcaggaggaaggaaaccttggcacagtatcaactttgtatgtgcacatgatggatttacactggctgatttggtaacatataataagaagtacaatttaccaaatggggagaacaacagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttctacatgggtgatgaatatggccacacaaaagggggcaacaacaatacatactgccatgattcttatgtcaattattttcgctgggataaaaaagaacaatactctgagttgcaccgattctgctgcctcatgaccaaattccgcaaggagtgcgagggtcttggccttgaggactttccaacggccaaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatacgacttcctcaccgacgacttacctgatcgcgctctcaccatacaccagttctcgcatttcctctactccaacctctaccccatgctcagctactcatcggtcatcctagtattgcgccctgatgtttgagagaccaatatatacagtaaataatatgtctatatgtagtcctttggcgtattatcagtgtgcacaattgctctattgccagtgatctattcgatccacagatacatgtgcaaactgcaaagtttctggtaatcagagaagtttttcctg

SEQ ID NO: 55 - DNA, 2574 bp, Triticum aestivum
ctcccacgaagaaccaacaggaggcgcggatcccaccgataaataaccccgcctcgccgctcctccccaaaatcaatcaccgatcgctcggggttcccggcatgacgatgatggccatggccaaggcgccctgcctctgcgcgcgcccgtccctcgccgcgcgcgcgaggcggccggggccggggccggcgccgcgcctgcgacggtggcgacccaatgcgacggcggggaagggggtcggcgaggtgtgcgccgcggttgtcgaggcggcgacgaaggccgaggatgaggacgacgacgaggaggaggcggtggcggaggacaggtacgcgctcggcggcgcgtgcagggtgctcgccggaatgcccgcgccgctgggcgccaccgcgctcgccggcggggtcaatttcgccgtctactccggtggagccaccgccgcggcgctctgcctcttcacgccagaagatctcaaggcggatagggtgacggaggaggtttcccttgaccccctgatgaatcggactgggaacgtgtggcatgtcttcattgaaggcgagctgcacgacatgctttacgggtacaggttcgacggcacctttgctcctcactgcgggcactaccttgatatttccaatgtcgtggtggatccttatgctaaggcagtgataagccgaggggagtatggcgttccggcgcgtggtaacaattgctggcctcagatggctggcatgatccctcttccatatagcacgtttgattgggaaggcgacctacctctaagatatcctcaaaaggacctggtaatatatgagatgcacttgcgtggattcacgaagcatgattcaagcaatgtagaacatccgggtactttcattggagctgtgtcgaagcttgactatttgaaggagcttggagttaattgtattgaattaatgccctgccatgagttcaacgagctggagtactcaacctcttcttccaagatgaacttttggggatattctaccataaacttcttttcaccaatgacgagatacacatcaggcgggataaaaaactgtgggcgtgatgccataaatgagttcaaaacttttgtaagagaggctcacaaacggggaattgaggtgatcctggatgttgtcttcaaccatacagctgagggtaatgagaatggtccaatattatcatttaggggggtcgataatactacatactatatgcttgcacccaagggagagttttataactattctggctgtgggaataccttcaactgtaatcatcctgtggttcgtcaattcattgtagattgtttaagatactgggtgatggaaatgcatgttgatggttttcgttttgatcttgcatccataatgaccagaggttccagtctgtgggatccagttaacgtgtatggagctccaatagaaggtgacatgatcacaacagggacacctcttgttactccaccacttattgacatgatcagcaatgacccaattcttggaggcgtcaagctcattgctgaagcatgggatgcaggaggcctctatcaagtaggtcaattccctcactggaatgtttggtctgagtggaatgggaagtaccgggacattgtgcgccaattcattaaaggcactgatggatttgctggtggttttgccgaatgtctttgtggaagtccacacctataccaggcaggaggaaggaaaccttggcacagtatcaactttgtatgtgcacatgatggatttacactgggtgatttggtaacatataataacaagtacaatttaccaaatggggagaacaatagagatggagaaaatcacaatcttagctggaattgtggggaggaaggagaattcgcaagattgtctgtcaaaagattgaggaagaggcagatgcgcaatttctttgtttgtctcatggtttctcaaggagttccaatgttttacatgggcgatgaatatggccacacaaaagggggcaacaacaatacatactgccatgattcttatgtcaattattttcgctgggataaaaaagaacaatactctgacttgcacagattctgctgcctcatgaccaaattccgcaaggagtgcgatggtcttggccttgaggactttccaacggccgaacggctgcagtggcatggtcatcagcctgggaagcctgattggtctgagaatagccgattcgttgccttttccatgaaagatgaaagacagggcgagatctatgtggccttcaacaccagccacttaccggccgttgttgagctcccagagcgcgcagggcgccggtgggaaccggtggtggacacaggcaagccagcaccatatgacttcctcaccgacgacttacctgatcgcgctctcaccatacaccagttctctcatttcctcaactccaacctctaccccatgctcagctactcatcggtcatcctagtattgcgccctgatgtttgagagacaaatatatacagtaaataatatgtctatatgtagtcctttggcgtattatcagtgtgcacaattgctctattgccagtgatctattcgatcc

SEQ ID NO: 56 - DNA, 376 bp, Triticum aestivum
ctattctggctgtgggaataccttcaactgtaatcatcctgtggttcgtcaattcattgtagattgtttaaggtacagatatacattttattctagaactacttttcatttctttcgctgcttgtcattttgatatgattaatttgcaagcttgtggggtaaatcttttggtcagcatattgtatctttaaatgtcacctaatgtcttggtgcttatagatttggcatcttcaaattctttttctccaatgaaaagggaaaaatctactgtatgtctggtcaactaatttacttttgttttgcagatactgggtgacggaaatgcatgttgaggttttcgttttgatcttgcatccataatgaccagaggttccag

SEQ ID NO: 57 - DNA, 382 bp, Triticum aestivum
ctattctggctgtgggaataccttcaactgtaatcatcctgtggttcgtcaattcattgtagattgtttaaggtacagatatacattttattctagaactacttttcatttctttcgctgcttgtcattttgatatgattaatttgcaagcttgtgggggtaaatcttttggtcagcatattgtatctttaaatgtcaccaatactaatgtcctggtgcttattgatttggcatcttcaaattctttttctccagtgaaaagggaaaaatctactgtatgtctcgtcaactaatttacttttattttgcagatactgggtgacggaaatgcatgttgaggttttcgttttgatcttgcatccataatgaccagaggttccag

SEQ ID NO: 58 - DNA, 380 bp, Triticum aestivum
ctattctggctgtgggaataccttcaactgtaatcatcctgtggttcgtcaattcattgtagattgtttaaggtacagatatacattttattctagaactactttttcatttcttttgctgcttgtcattttgatatgattaatttgcaagcttgtgggggtaaatcttttggtcagcatattgtatctttaaatgtcacaaatactaatgtcctggtgcttattgatttggcatcttcaaattcttctccaatgaaaagggaaaaatctactgtatgtctcgtcaactaatttacttttgttttgcagatactgggtgatggaaatgcatgttgaggttttcgttttgatcttgcatccataatgaccagaggttccag

SEQ ID NO: 59 - DNA, 490 bp, Triticum aestivum
cttcacgccagaagatctcaaggcggtggggttgcctcccgagtagagttcatcagctttgcgtgcgccgcccgccccctttttggcctgcgatttgagttttgtatcgggggaaatgctgcaggatagggtgacggaggaggttccccttgaccccctgatgaatcggactgggaacgtatggcatgtcttcatcgaaggcgagctgcaggacatgctttacgggtacaggttcgacggcacctttgctcctcactgcgggcactaccttgatgtttccaatgtcgtggtggatccttatgctaaggtgatcatacattagctttacctgcatcttggtatttacagtagaaattgttacgcggacgcttatttgctgccttttgtgttgctctaggcagtgataagccgaggggagtatggtgttccggcgcgtggtaacaattgctggcctcagatggctggcatgatccctcttccatatagcacg

SEQ ID NO: 60 - DNA, 491 bp, Triticum aestivum
cttcacgccagaagatctcaaggcggtggggttgcctcccgagtagagttcatcagctttgcgtgcgccgcgcgcccccttttctggcctgcgatttaagttttgtactgggggaaatgctgcaggatagggtgacggaggaggtttcccttgaccccctgatgaatcggactgggaacgtgtggcatgtcttcattgaaggcgagctgcacgacatgctttacgggtacaggttcgacggcacctttgctcctcactgcgggcactaccttgatatttccaatgtcgtggtggatccttatgctaaggtgatcatactttagctttacctgcatcttggtatttacagtagaaattgttacgtggacccttatttgttgccttttgtgttgctctaggcagtgataagccgaggggagtatggcgttccggcgcgtggtaacaattgctggcctcagatggctggcatgatccctcttccatatagcacg

SEQ ID NO: 61 - DNA, 491 bp, Triticum aestivum; identical to SEQ ID NO: 36

SEQ ID NO: 62 - DNA, 22 bp, Triticum aestivum
cttcacgccagaagatctcaag

SEQ ID NO: 63 - DNA, 22 bp, Triticum aestivum
cgtgctatatggaagagggatc

SEQ ID NO: 64 - DNA, 23 bp, Triticum aestivum
gaggtgatcatggatgttgtctt

SEQ ID NO: 65 - DNA, 23 bp, Triticum aestivum
agtatagatgcaaggtcaaagca

SEQ ID NO: 66 - protein, 20 aa, Triticum aestivum
AAVVEAATKAEDEDDDDEEE

SEQ ID NO: 67 - DNA, 22 bp, Triticum aestivum
ctattctggctgtgggaatacc